(19) United States
(12) Patent Application Publication
(10) Pub. No.: US 2010/0069643 A1
(43) Pub. Date: Mar. 18, 2010

(54) Convergent Process for the Synthesis of Taxane Derivatives

(76) Inventors: James D. McChesney, Boulder, CO (US); John T. Henri, Longmont, CO (US); Sylesh K. Venkataraman, Longmont, CO (US); Rodger L. Lamb, Westminster, CO (US); Jonathan E. Foster, Boulder, CO (US); Christian M. Summer, Boulder, CO (US); Shangping Ye, Florence, SC (US)

Correspondence Address: MORGAN, LEWIS & BOCKIUS LLP, 1701 MARKET STREET, PHILADELPHIA, PA, 19103-2921, US

(21) Appl. No.: 12/158,473

(22) PCT Filed: Dec. 21, 2006
(86) PCT No.: PCT/US2006/048759

§ 371 Date: Sep. 28, 2009

Related U.S. Application Data
(60) Provisional application No. 60/786,629, filed on Mar. 27, 2006.

(30) Foreign Application Priority Data
Dec. 21, 2005 (US) PCTUS2005046887

(51) Int. Cl. C07D 305/14; C07D 263/06; C07C 69/96; C07D 413/12; C07D 493/04

(52) U.S. Cl. 548/215; 558/276; 549/510; 549/358

(57) ABSTRACT

The application provides a process for the preparation of taxane derivatives and intermediates useful in such processes.

### CROSS REFERENCE TO RELATED APPLICATIONS

This application claims the benefit of priority under 35 U.S.C. 120 of International PCT Application No. PCT/US05/46887, filed Dec. 21, 2005, entitled “Novel Compounds and Methods for Forming Taxanes and Using the Same”, and claims the benefit of U.S. Provisional Application No. 60/786,629, filed Mar. 27, 2006, both of which are currently pending.

### FIELD OF THE APPLICATION

The present application generally relates to process for the preparation of taxane derivatives useful in the treatment of cancer in patients and to intermediates useful in such processes. More particularly this application relates to processes useful inter alia in the preparation of paclitaxel, docetaxel and to certain 9,10-α,α-OH taxane analogues having a bridge between the 7-OH and 9-OH groups.

### BACKGROUND TO THE APPLICATION

Paclitaxel and docetaxel are well established anticancer agents for which numerous synthetic methods are known. Methods of synthesis of certain 9,10-α,αOH taxane analogues are disclosed in WO 2005/030152. Other synthetic methods are disclosed in EP 1 228 759 A (Florida State University), EP 1 285 920 A (Florida State University), EP 1 148 055 A (Florida State University), WO 01/56564 A (Florida State University), WO 01/57027 (Florida State University), WO 94/10996 A (Florida State University), FR 2 715 846 A (Rhone-Poulenc), U.S. Pat. No. 5,352,806 A, FR 2 707 293 A (Rhone-Poulenc), WO 94/08984 A (Rhone-Poulenc), WO 92/09589 A (Rhone-Poulenc), WO 94/20485A (Florida State University), WO 93/21173 A (Abbott), Klein L L: “Synthesis of 9-Dihydrotaxol: A Novel Bioactive Taxane” Tetrahedron Letters., Elsevier, Amsterdam, NL, vol 34, no 13, 1993, pp 2047-2050, Datta A et al: “Synthesis of Novel C-9 and C-10 modified bioactive taxanes” Tetrahedron Letters, Elsevier, Amsterdam, NL, Vol 36, no 12, 20 Mar. 1995, pp 1985-1988, Klein L L et al: Journal of Medicinal Chemistry, American Chemical Society. Washington, no 38, 1995, pp 1482-1492, J. Demattei et al: “an efficient synthesis of the taxane-derived anticancer agent abt-271”, Journal of Organic Chemistry, vol 66, no 10, 2001, pp 3330-3337, Gunda I. Georg et al: “the chemistry of the taxane diterpene: stereoselective reductions of taxanes” Journal of organic chemistry, vol 63, no 24, 1998, pp 8926-8934, U.S. Pat. No. 4,924,011, U.S. Pat. No. 5,015,744, U.S. Pat. No. 6,107,497, U.S. Pat. No. 5,770,745 and U.S. Pat. No. 5,750,737.

Many syntheses of paclitaxel and docetaxel and other taxane derivatives involve the use of a β-lactam to acylate the β-hydroxy group of a 10-baccatin III or a 10-deacetylbaccatin III derivative. Other methods have described the coupling of a carboxylic acid to 10-baccatin III or a 10-deacetylbaccatin III, for example with DCC. WO 2005/03150 discloses an improved process for coupling certain side chains to the β-hydroxy group of taxane variants by using an acyl fluoride.

The formula of paclitaxel is:

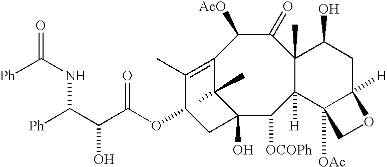

The formula of docetaxel is:

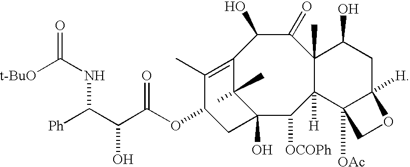

The formula of TPI287:

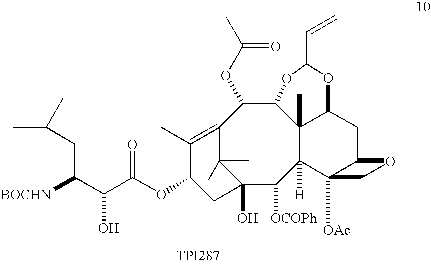

The numbering system of the taxane backbone is:

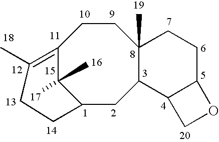

Since paclitaxel appears promising as a chemotherapeutic agent, chemists have spent substantial time and resources in attempting to synthesize paclitaxel and other potent taxane analogs. The straightforward implementation of the partial synthesis of paclitaxel or other taxanes, requires convenient access to chiral, non-racemic side chains and derivatives, an abundant natural source of baccatin III or closely related diterpenoid substances, and an effective means of joining the two units. Perhaps the most direct synthesis of paclitaxel is the condensation of Baccatin III and 10-deacetylbaccatin III of the formula:

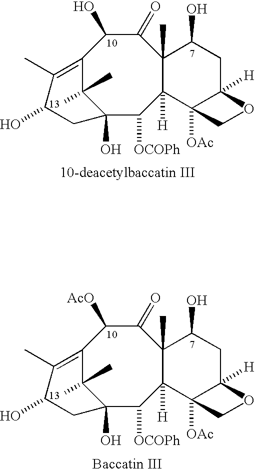

with the side chain:

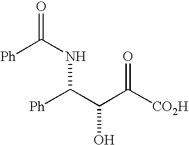

However, the esterification or coupling of these two units is difficult, because of the C-13 hydroxyl group of both baccatin III and 10-deacetylbaccatin III are located within the sterically encumbered concave region of the hemispherical taxane skeleton.

Alternative methods of coupling the side chain to a taxane backbone to ultimately produce paclitaxel have been disclosed in various patents. For example, U.S. Pat. No. 4,929,011 issued May 8, 1990 to Denis et al. entitled “Process for Preparing Taxol”, describes the semi-synthesis of paclitaxel from the condensation of a (2R,3S) side chain acid of the general formula:

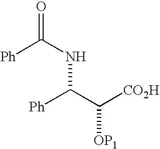

wherein P₁ is a hydroxyl protecting group, with a taxane derivative of the general formula:

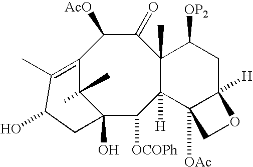

wherein P₂ is a hydroxyl protecting group. The condensation product is subsequently processed to remove the P₁ and P₂ protecting groups. In Denis et al., the paclitaxel C-13 side chain, (2R,3S)3-phenylisoserine derivative is protected with P₁ for coupling with a protected Baccatin III. The P₂ protecting group on the baccatin III backbone is, for example, a trimethylsilyl or a trialkylsilyl radical.

An alternative semi-synthesis of paclitaxel is described in U.S. Pat. No. 5,770,745 to Swindell et al. Swindell et. al. disclose semi-synthesis of paclitaxel from a baccatin III backbone by the condensation with a side chain having the general formula:

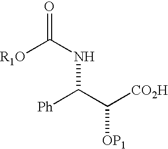

wherein R₁ is alkyl, olefinic or aromatic or PhCH₂ and P₁ is a hydroxyl protecting group.

Another method for the semi-synthesis of paclitaxel is found in U.S. Pat. No. 5,750,737 to Sisti et al. In this patent, C7-CBZ baccatin III of the formula

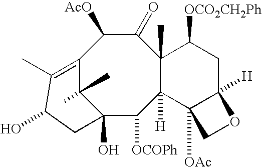

is esterified with a C3-N—CBZ—C2-O-protected (2R,3S)-3-phenylisoserine side chain of the formula:

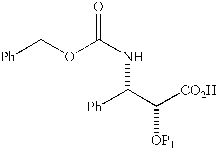

followed by deprotection, and C3′N benzoylation to produce paclitaxel.

As noted above, docetaxel is similar to paclitaxel except for the t-butoxycarbonyl (t-Boc) group at the C3′ nitrogen position of the phenylisoserine side chain and a free hydroxyl group at the C10 position. Similar to paclitaxel, the synthesis of docetaxel is difficult due to the hindered C13 hydroxyl in the baccatin III backbone, which is located within the concave region of the hemispherical taxane skeleton. Several syntheses of docetaxel and related compounds have been reported in the Journal of Organic Chemistry: 1986, 51, 46; 1990, 55, 1957; 1991, 56, 1681; 1991, 56, 6939; 1992, 57, 4320; 1992, 57, 6387; and 993, 58, 255; also, U.S. Pat. No. 5,015,744 issued May 14, 1991 to Holton describes such a synthesis. Additional techniques for the synthesis of docetaxel are discussed, for example, in U.S. Pat. No. 5,688,977 to Sisti et al. and U.S. Pat. No. 6,107,497 to Sisti et al.

While the existing procedures for synthesizing paclitaxel, docetaxel and TPI 287 have merit, there is still a need for improved chemical processes for preparing these anti-cancer compounds and their derivatives in good yields. The present application is directed to meeting these needs.

### SUMMARY OF THE APPLICATION

This application provides an effective synthesis of taxane derivatives by esterifying a 13-OH group of a taxane derivative with a cyclically protected side chain acid and thereafter removing the protecting groups.

Thus the last two steps of the process may be represented as a process for the preparation of a compound of the formula (VIII):

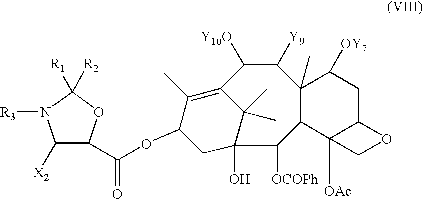

which comprises the reaction of a compound of the formula (IX):

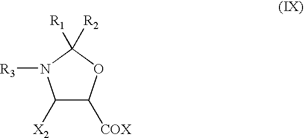

with a compound of the formula (X):

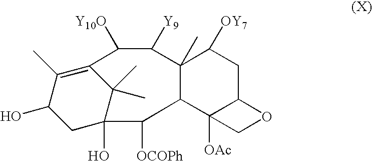

wherein:
R₁ and R₂ are independently H or substituted or unsubstituted alkyl, alkenyl, aryl, aralkyl or acyl;
R₃ is H or P₁ where P₁ is an amino protection group;
X is halogen or OR⁴ where R⁴ is H, a substituted or unsubstituted alkyl, alkenyl, aryl, aralkyl, acyl, acyloxycarbonyl or aryloxycarbonyl;
X₂ is substituted or unsubstituted alkyl, alkenyl, aryl, aralkyl or acyl;

### Y₇ is R₇, P₃ or Z₇;

Y₉ is H, OH, a ketone, OR₉, P₄ or Z₉;

### Y₁₀ is R₁₀, P₅ or Z₁₀;

R₇ is H, substituted or unsubstituted alkyl, alkenyl, aryl, aralkyl or acyl;
Z₇ is P₃ and together with Y₉ forms a cyclic structure when Y₉ is P₄;
Z₉ is either R₉ and together with Y₇ forms a cyclic structure when Y₇ is P₃; or
Z₁₀ is P₅ and together with Y₉ forms a cyclic structure when Y₉ is P₄;
P₅ and together with Y₁₀ forms a cyclic structure when Y₁₀ is P₄;
R₉ is a substituted or unsubstituted alkyl, alkenyl, aryl, aralkyl or acyl;
R₁₀ is H, substituted or unsubstituted alkyl, alkenyl, aryl, aralkyl or acyl;
P₃ is a hydroxyl protecting group;
P₄ is a hydroxyl protecting group; and
P₅ is a hydroxyl protecting group
and thereafter removing the side chain protecting groups and optionally other protecting groups to provide a compound of the formula (XVI):

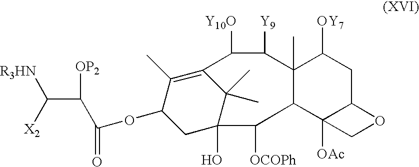

Advantageously, if there is more than one protecting group on the taxane nucleus, they can be removed simultaneously if required.

Here, if desired, X is a halogen; X₂ is Ph; Y₇ is P₃; Y₉ is a ketone; Y₁₀ is P₅; R₁ is H; P₁ is Boc; P₂ is BOM; P₃ is Cbz; and P₅ is Cbz. Alternatively, X is fluorine; X₂ is Ph; Y₇ is P₃; Y₉ is a ketone; Y₁₀ is P₅; R₁ is H; P₁ is Cbz; P₂ is BOM; P₃ is Cbz; and P₅ is Cbz. In another alternative, X is OR₄; X₂ is isobutyl; Y₇ is P₃; Y₉ is a ketone; Y₁₀ is P₅; R₁ is H; R₄ is H; P₁ is Boc; P₂ is BOM; P₃ is Cbz; and P₅ is Cbz. In yet another alternative, X is a halogen; X₂ is isobutyl; Y₇ is P₃; Y₉ is a ketone; Y₁₀ is P₅; R₁ and R₂ are independently H or substituted or unsubstituted: alkyl, alkenyl, aryl, aralkyl, or acyl; R₃ is H; P₁ is Boc; P₂ is BOM; P₃ is Cbz; and P₅ is Cbz. The benzoxycarbonyl group is frequently a preferred protecting group to use.

The application also provides particularly apt protected side chain acids for use in the process. These are of the formula (I):

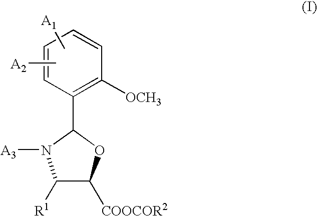

wherein A₁ is hydrogen, halogen, lower alkyl or lower alkoxy;
A₂ is hydrogen, halogen, lower alkyl or lower alkoxy;

### A₃ is BOC; Cbz or PhCO;

R¹ is lower alkyl or phenyl, and

R² is an alkyl or an aryl group such that the moiety OCOR² is readily displaced from the compound of formula (I) by an alcohol or alkoxide.

As used above, and throughout the description of the application, the following terms, unless otherwise indicated, shall be understood to have the following meanings:

The term “alkyl” as used herein alone or as part of another group, denotes optionally substituted, straight and branched chain saturated hydrocarbon groups, preferably having 1 to 12 carbons in the normal chain.

The term “substituted alkyl” refers to an alkyl group substituted by, for example, one to four substituents, such as, halo, trifluoromethyl, trifluoromethoxy, hydroxy, alkoxy, cycloalkyoxy, heterocyclooxy, oxo, alkanoyl, aryloxy, alkanoyloxy, amino, alkylamino, arylamino, aralkylamino, cycloalkylamino, heterocycloamino, disubstituted amines in which the 2 amino substituents are selected from alkyl, aryl or aralkyl, alkanoylamino, aroylamino, aralkanoylamino, substituted alkanoylamino, substituted arylamino, substituted aralkanoylamino, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, sulfonamido (e.g. SO₂NH₂), substituted sulfonamido, nitro, cyano, carboxy, carbamyl (e.g. CONH₂), substituted carbamyl (e.g. CONH alkyl, CONH aryl, CONH aralkyl or cases where there are two substituents on the nitrogen selected from alkyl, aryl or aralkyl), alkoxycarbonyl, aryl, substituted aryl, guanidino and heterocyclos, such as, indolyl, imidazolyl, furyl, thienyl, thiazolyl, pyrrolidyl, pyridyl, pyrimidyl and the like. Where noted above where the substituent is further substituted it will be with halogen, alkyl, alkoxy, aryl or aralkyl. Exemplary unsubstituted such groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl and the like. Exemplary substituents may include one or more of the following groups: halo, alkoxy, alkylthio, alkenyl, alkynyl, aryl, cycloalkyl, cycloalkenyl, hydroxy or protected hydroxy, carboxyl (—COOH), alkyloxycarbonyl, alkylcarbonyloxy, carbamoyl (NH₂CO—), amino (—NH₂), mono- or dialkylamino, or thiol (—SH).

The term “alkenyl”, as used herein alone or as part of another group, denotes such optionally substituted groups as described for alkyl, further containing at least one carbon to carbon double bond. Exemplary substituents include one or more alkyl groups as described above, and/or one or more groups described above as alkyl substituents.

The term “aryl”, as used herein alone or as part of another group, denotes optionally substituted, homocyclic aromatic groups, preferably containing 1 or 2 rings and 6 to 12 ring carbons. Exemplary unsubstituted such groups include phenyl, biphenyl, and naphthyl. Exemplary substituents include one or more; preferably three or fewer, nitro groups, alkyl groups as described above, and/or groups described above as alkyl substituents.

The term “substituted aryl” refers to an aryl group substituted by, for example, one to four substituents such as alkyl; substituted alkyl, halo, trifluoromethoxy, trifluoromethyl, hydroxy, alkoxy, cycloalkyloxy, heterocyclooxy, alkanoyl, alkanoyloxy, amino, alkylamino, aralkylamino, cycloalkylamino, heterocycloamino, dialkylamino, alkanoylamino, thiol, alkylthio, cycloalkylthio, heterocyclothio, ureido, nitro, cyano, carboxy, carboxyalkyl, carbamyl, alkoxycarbonyl, alkylthiono, arylthiono, alkysulfonyl, sulfonamido, aryloxy and the like. The substituent may be further substituted by halo, hydroxy, alkyl, alkoxy, aryl, substituted aryl, substituted alkyl or aralkyl.

The term “aralkyl”, as used herein alone or as part of another group refers to alkyl groups as discussed above having an aryl substituent, such as benzyl or phenethyl, or naphthylpropyl, or an aryl as defined above.

The term “acyl”, as used herein alone or as part of another group, denotes the moiety formed by removal of the hydroxyl group from the group —COOH of an organic carboxylic acid. The acyl group can specifically be PhCO or BnCO, for example.

The term “hydroxy (or hydroxyl) protecting group”, as used herein, denotes any group capable of protecting a free hydroxyl group which, subsequent to the reactions for which it is employed, may be removed without destroying the remainder of the molecule. Such groups, and the synthesis thereof, may be found in “Protective Groups in Organic Synthesis” by T. W. Greene and P. G. M. Wuts, Protective Groups in Organic Synthesis, 3rd Edition, John Wiley & Sons, New York (1999), or Fieser & Fieser. Exemplary hydroxyl protecting groups include methoxymethyl, 1-ethoxyethyl, 1-methoxy-1-methylethyl, benzyloxymethyl, (β-trimethylsilyl-ethoxy)methyl, tetrahydropyranyl, benzyloxycarbonyl, 2,2,2-tri-chloroethoxycarbonyl, t-butyl(diphenyl)silyl, trialkylsilyl, trichloromethoxycarbonyl, and 2,2,2-trichloroethoxymethyl.

The term “amine protecting group” as used herein means an easily removable group which is known in the art to protect an amino group against undesirable reaction during synthetic procedures and to be selectively removable. The use of amine protecting groups is well known in the art for protecting groups against undesirable reactions during a synthetic procedure and many such protecting groups are known, for example, T. W. Greene and P. G. M. Wuts, Protective Groups in Organic Synthesis, 3rd Edition, John Wiley & Sons, New York (1999), incorporated herein by reference. Exemplary amine protecting groups are acyl, including formyl, acetyl, chloroacetyl, trichloroacetyl, O-nitrophenylacetyl, o-nitrophenoxyacetyl, trifluoroacetyl, acetoacetyl, 4-chlorobutyryl, isobutyryl, o-nitrocinnamoyl, picolinoyl, acylisothiocyanate, aminocaproyl, benzoyl and the like, and acyloxy including methoxycarbonyl, 9-fluorenylmethoxycarbonyl, 2,2,2-trifluoroethoxycarbonyl, 2-trimethylsilylethoxycarbonyl, vinyloxycarbonyl, allyloxycarbonyl, t-butyloxycarbonyl (BOC), 1,1-dimethylpropynyloxycarbonyl, benzyloxycarbonyl (CBZ), p-nitrobenzyloxycarbonyl, 2,4-dichlorobenzyloxycarbonyl, and the like.

The term “halogen” as used herein alone or as part of another group, denotes chlorine, bromine, fluorine, and iodine of which fluorine and chlorine are preferred.

### DETAILED DESCRIPTION OF THE APPLICATION

This application provides the compounds of the formula (I)

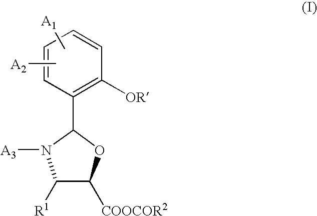

wherein A₁ is hydrogen, halogen, lower alkyl or lower alkoxy;
A₂ is hydrogen, halogen, lower alkyl or lower alkoxy;

### A₃ is BOC, Cbz or COPh;

R′ is methyl, ethyl or lower alkyl (C₁ through C₆)
R¹ is lower alkyl or phenyl group, and
R² is an alkyl, aralkyl or an aryl group such that the moiety OCOR² is readily displaced from the compound of formula (I) by an alcohol or alkoxide.

The compounds of formula (I) have been found to be particularly effective agents for esterifying the 13-OH group of the taxane nucleus. Additionally, it has been found that removal of the protective moiety after coupling can be carried out without causing undesired epimerization of the side chain, for example by hydrogenation, for example employing a palladium or charcoal catalyst.

It is also an advantage that, for example in the synthesis of paclitaxel or docetaxel, that a protecting group on the 7-hydroxy and/or 10-hydroxy group can be conveniently removed, for example if they are a Cbz or another hydrogenolysable group.

The term “lower” means up to 6 carbon atoms, more aptly up to 4 carbon atoms. Hence lower alkyl can be methyl to hexyl, more aptly methyl, ethyl, propyl or butyl and is preferably methyl. Similarly lower alkoxy can be methoxy to hexyloxy, more aptly methoxy to butoxy and preferably methoxy.

The most apt halogens are chloride and fluorine of which fluorine is preferred.

In compounds of formula (I), A₂ is preferably a hydrogen atom.

In compounds of formula (I), A₁ is favourably a methoxy group, especially a 4- or 6-methoxy group and preferably a 6-methoxy group.

In some compounds of formula (I), R¹ is favorably an iso-butyl group i.e. a (CH₃)₂ CHCH₂— group.

In other compounds of formula (I), R¹ is favourably a phenyl group.

Hence certain particularly apt compounds of the formula (I) are those of the formulae (II), (III), (IV) and (V):

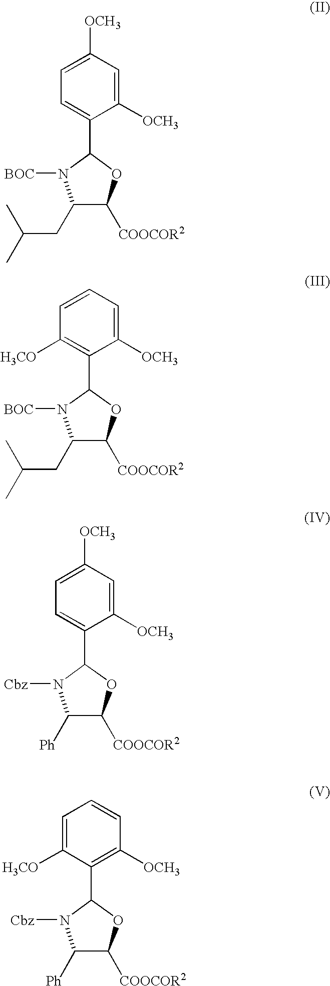

In compounds of the formulae (I), (II), (III), (IV) and (V), R² is a group such that OCOR² is readily displaceable on reaction with an hydroxy group or metal alkoxide group. Thus the 13-OH group of the taxane derivative does not become acylated to any wasteful extent by acylation by the COR² moiety when the compound of the formulas (I), (II), (III), (IV) or (V) is employed as an acylating agent.

An acid chloride or acid fluoride analogous to the above mixed compounds may also be employed but this has been found to be less advantageous than the use of the above mixed anhydrides.

A particularly preferred group R² is the t-butyl group. This can be prepared from the corresponding acid and pivaloyl chloride (CH₃)₃CCOCl, for example in situ prior to the acylation of the 13-OH group.

Other R² groups include lower alkyl, lower aralkyl and aryl groups. The group R² is aptly a voluminous group, for example a tertiary alkyl group or an electron withdrawing group so that the ⁻OCOR² anion is stabilised. Thus apart from the t-butyl group, other apt groups R² include benzhydril, trityl, phenyl, 2,4-dichlorophenyl, 2,6-dichlorophenyl, 2,6-di-tert-butyl-phenyl, 4-nitrophenyl and the like.

The compounds of the formula (VI):

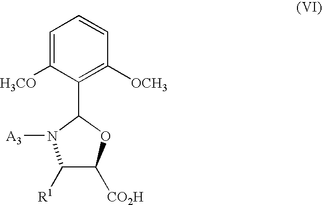

is a particularly favoured acid for use in the preparation of the compounds of the formula (I).

The acids corresponding to compounds of the formula (I) may be prepared by the oxidation of a compound of the formula (VII):

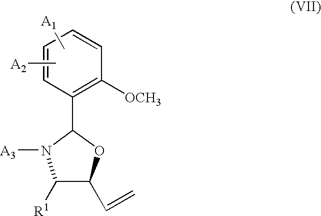

wherein A₁, A₂, A₃ and R¹ are as defined in relation to formulas (I)-(V). This may be effected by conventional mild vinylic oxidation reagents for example. NaIO₄, OsO₄, NMO, TPAP, ozone etc.

The compound of the formula (VII) may be prepared by the following sequence:

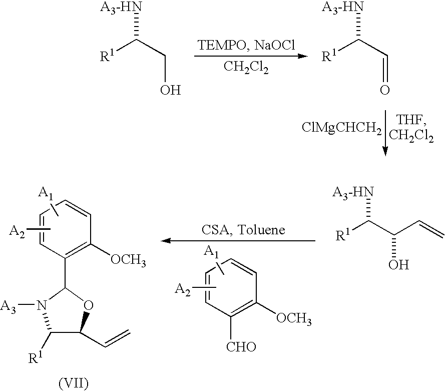

The reaction of vinyl magnesium chloride with the aldehyde in a mixture of tetrahydrofuran leads to a 4:1 diastereomeric ratio of products with the desired isomer predominating. This desired isomer reacts preferentially with the substituted benzaldehyde to provide the compound of the formula (VII).

This process is particularly useful in the preparation of the compound of formula (VI) by using 2,6-dimethoxybenzaldehyde.

The compound of the formula (VI) (and other carboxyl acids as referred to above) may be converted into the acid chloride or acid fluoride in conventional manner. A particularly favoured method of preparing an acid fluoride is by reaction with (CH₃CH₂)₂N—SF₃ or deoxofluor, for example in pyridine and dichloromethane. Such acyl fluorides may be, reacted with the 13-OH group of a taxane derivative in, for example, dichloromethane or tetrahydrofuran with a base, for e.g., DMAP, DBU etc. (However, as previously indicated, it is preferred to prepare and employ a compound of the formula (I) in situ (i.e. without isolation of the compound of formula (I) prior to its use) to acylate the 13-OH group of the taxane derivative).

The preparation of the compound of formula (I) from the analogous acid is generally carried out under an inert atmosphere, for example nitrogen, at a non-extreme temperature, for example an ambient temperature of 15-25° C. A non-hydroxylic solvent is employed, for example, tetrahydrofuran. A tertiary amine, for example N-methylmorpholine, is employed as proton acceptor. After dissolving the 13-OH taxane derivative and the acid in the solvent, pivaloyl chloride (or other compound of the formula ClCOR²) is added and the reaction mixture allowed to stir at ambient temperature until complete (as indicated by HPLC).

In order to remove the protecting group or groups conventional conditions are employed. Thus for example, if only the side chain protecting group is to be removed, dilute acid may be employed. Suitably the solution may be cooled to about −18° C. to −20° C. and 0.5N HCL in methanol employed. The mixture may then be stirred at a depressed temperature, for example −15° C., until deprotection is complete (as indicated by HPLC). The reaction may then be quenched, for example 5% sodium bicarbonate solution) and concentrated by evaporation before yielding the product. Alternatively if it is also desired to remove a protecting group from, for example the 7-OH and (or 10-OH group of a taxane derivative, then methods of deprotection can be employed which deprotect both the side chain and the backbone hydroxyl groups. Thus, if it is wished to employ acidic deprotection, then the backbone hydroxyl group(s) can be protected by using a protecting group which is readily cleavable with acid so that a single deprotecting reagent is employed. Also, the backbone hydroxyl group(s) can be protected with a group readily removable by hydrogenation, for example a Cbz group. In such circumstances hydrogenation may be effected in conventional manner, for example employing 10% Pd/C catalyst in a THF, an aqueous THF or methanolic solution, followed by an acidification, for example with formic or acetic acid, for example in methanol. Such a hydrogenation reaction could be employed when A₃ is a Cbz group so that a deprotected primary amino group could be produced in the side chain which could be thereafter acylated to provide a benzoyl or BOC substituted amino group if desired.

It will be understood that particularly, apt intermediates, even if not necessarily isolated, provided by this application are of the formula (XVII):

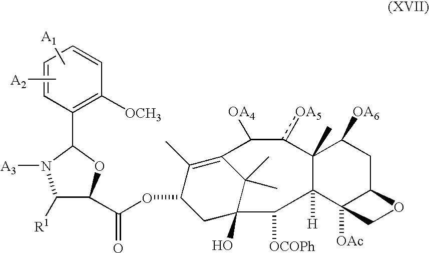

Favoured compounds of the formula (XVII) include those wherein A⁴ is a hydrogen atom or is a hydroxyl protecting group selected from the groups consisting of benzyl, Cbz or acetyl group, preferably an acetyl and A⁴ is in the α configuration, and A⁵ is joined to A₆ to form an O—CH(CH═CH₂)—O moiety and A₅ is in the α configuration.

Further favoured compounds of the formula (XVII) include those wherein OA₄ is in the β configuration and A₄ is hydrogen, Cbz or acetyl; A₅ is an oxo group; and A₆ is hydrogen or Cbz.

Compounds of the formula (XVII) are particularly apt for use in the preparation of paclitaxel, docetaxel or TPI287. Such compounds may be deprotected by acidification. Such compounds containing a Cbz group may be deprotected by hydrogenation. If such hydrogenation replaces a A₃ group which is Cbz by hydrogen, that compound may be acylated to yield one which contains a PhCO or BOC group, for example by reaction with the appropriate anhydride or acyl halide.

It will be appreciated that in a broad aspect this application provides a process for the preparation of a compound of the formula VIII:

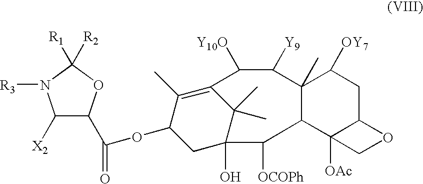

which comprises the reaction of a compound of the formula (IX):

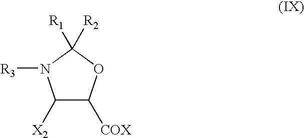

with a compound of the formula (X):

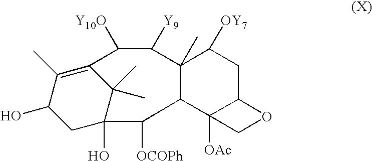

wherein:
R₁ and R₂ are independently H or substituted or unsubstituted alkyl, alkenyl; aryl, aralkyl or acyl;
R₃ is H or P₁ where P₁ is an amino protection group;
X is halogen or OR⁴ where R⁴ is H, a substituted or unsubstituted alkyl, alkenyl, aryl, aralkyl, acyl, acyloxycarbonyl or aryloxycarbonyl;
X₂ is substituted or unsubstituted alkyl, alkenyl, aryl, aralkyl or acyl;

### Y₇ is R₇, P₃ or Z₇;

Y₉ is H, OH, a ketone, OR₉, P₄ or Z₉;

### Y₁₀ is R₁₀, P₅ or Z₁₀;

R₇ is H, substituted or unsubstituted alkyl, alkenyl, aryl, aralkyl or acyl;
Z₇ is P₃ and together with Y₉ forms a cyclic structure when Y₉ is P₄;
Z₉ is either R₉ and together with Y₇ forms a cyclic structure when Y₇ is P₃; or
Z₁₀ is P₅ and together with Y₉ form a cyclic structure when Y₉ is P₄
P₅ and together with Y₉ forms a cyclic structure when Y₁₀ is P₄;
R₉ is a substituted or unsubstituted alkyl, alkenyl, aryl, aralkyl or acyl;
R₁₀ is H, substituted or unsubstituted alkyl, alkenyl, aryl, aralkyl or acyl;
P₃ is a hydroxyl protecting group;
P₄ is a hydroxyl protecting group; and
P₅ is a hydroxyl protecting group.

One skilled in the art will understand that the stereochemistry in taxane derivatives is defined as a result of their source from natural products except for the stereochemistry of positions 9 and 10 when synthetically amended. Hence that the compound of the formula (X) is more fully shown as:

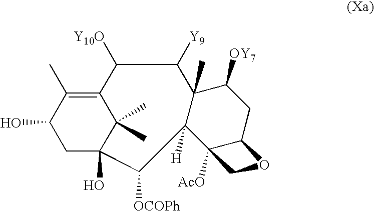

Similarly the skilled worker will understand from the stereochemistry of the side chain in paclitaxel, docetaxel and TPI 287 that the stereochemistry of the compound of the formula (IX) is more fully shown as:

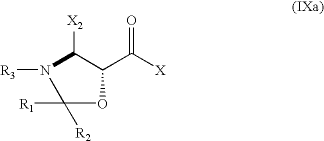

From the foregoing, the skilled worker will understand that the stereochemistry of formula (VIII) is fully shown as:

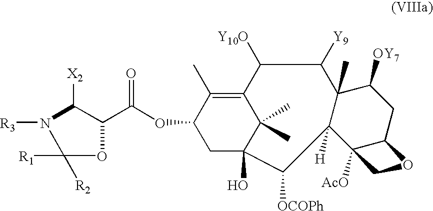

One skilled in the art would appreciate that the stereochemistry of the compound of formula (XVI) is more fully shown as:

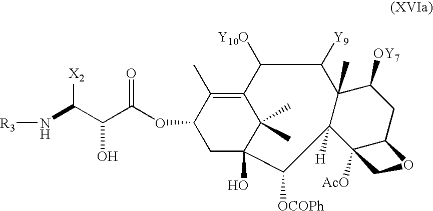

In this process, X may be fluorine or chlorine but is preferably OCOR² as defined in relation to formula (I), in particular a OCOC(CH₃)₃ group. In certain applications X may be a leaving group such as bromine, azide etc.

In the aforementioned process, the compound of formula (IX) is aptly of the formula (XI):

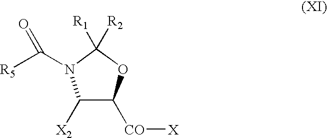

wherein X₂ is phenyl or CH₂CH(CH₃)₂;

### R⁵ is (CH₃)₃CO, Ph or PhO;

R₁ and R₂ are independently hydrogen, lower alkyl, lower alkyl substituted by lower alkoxy, phenyl or phenyl substituted by one, two or three groups selected from lower alkyl, lower alkoxy, fluorine or chlorine.

Aptly in formula (XI) R₁ is hydrogen. Aptly R₂ is an optionally substituted phenyl group. Preferably R₂ is a group of the formula

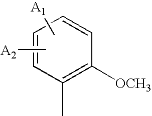

wherein A₁ and A₂ are as defined above.

In such process, the compound of the formula (X) is aptly of the formula (XII) or (XIII):

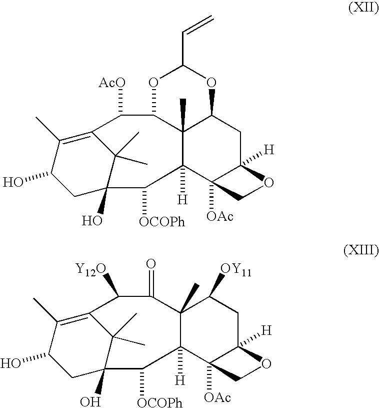

wherein Y₁₁ is hydrogen or a hydroxyl protecting group, such as a Cbz group and Y₁₂ is a hydrogen atom or a protecting group such as a Cbz or acetyl group.

In such process Y₁₁ is favourably a group removable by hydrogenation and is preferably a Cbz group.

Of the compounds according to formula (VIII) according to this application, favoured compounds are those of the formulae (XIV)

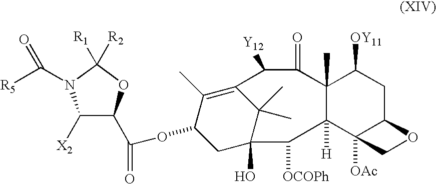

wherein R₁, R₂, R₅, X₂, Y₁₁ and Y₁₂ are as defined herein before. Such compounds are useful as intermediates in the synthesis of paclitaxel and docetaxel.

Compounds according to this application useful in the synthesis of TPI287 includes those of the formula (XV):

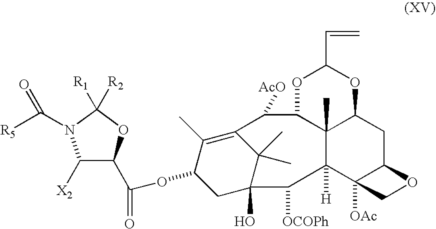

wherein R₁, R₂, R₅ and X₂ are as defined above.

This application also provides a process for the preparation of compounds of the formula (XVI)

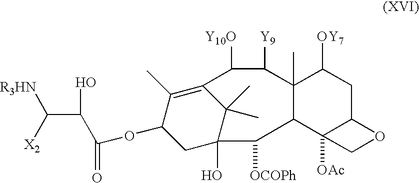

wherein X₂, R₃, Y₁₀, Y₉ and Y₇ are as defined in relation to formula (VIII) which comprises deprotection of the side chain moiety in the compounds of formula (VIII), preferably by treatment with acid, for example formic acid, acetic acid or aqueous HCl in methanolic solution. The skilled worker will appreciate the stereochemistry of the compound of formula (VIII) is more fully Shown as hereinbefore set out.

From the foregoing it will be appreciated that the present application provides a new and convergent synthesis for the preparation of taxane derivatives. This is illustrated with respect to TPI287 (shown as 10 in FIG. 1, other compounds with underlined numbers hereinafter also relate to the compounds of the sequence shown in FIG. 1). The process provides the desired products in high overall yields, requiring low numbers of chemical and mechanical processing steps, provides the desired compounds in high chemical purity and avoids the need for reverse phase purification and normal phase purification. Side chain deprotection uses mild, acid hydrolysis and avoids loss of product due to epimerization.

Suitable sequences are shown in FIGS. 1, 2 and 3:

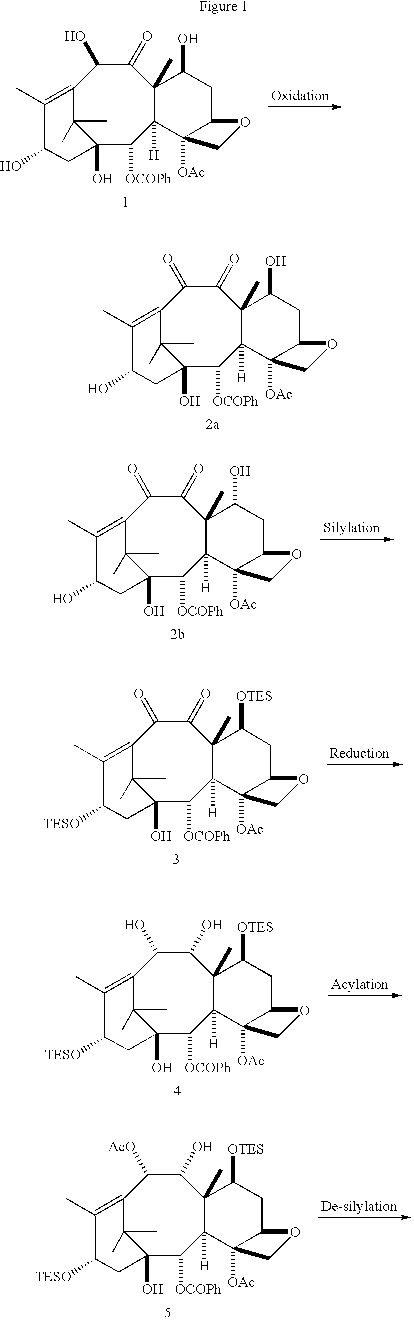

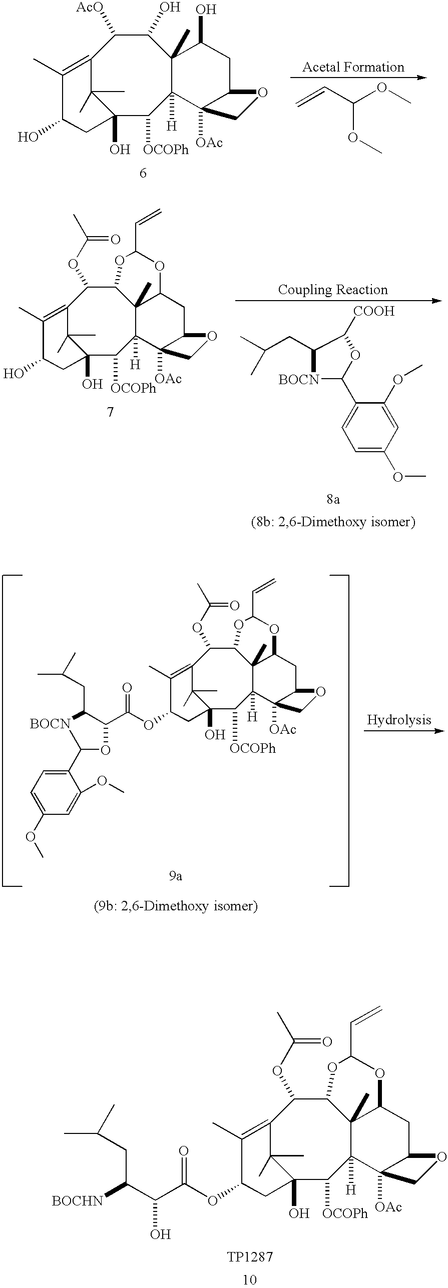

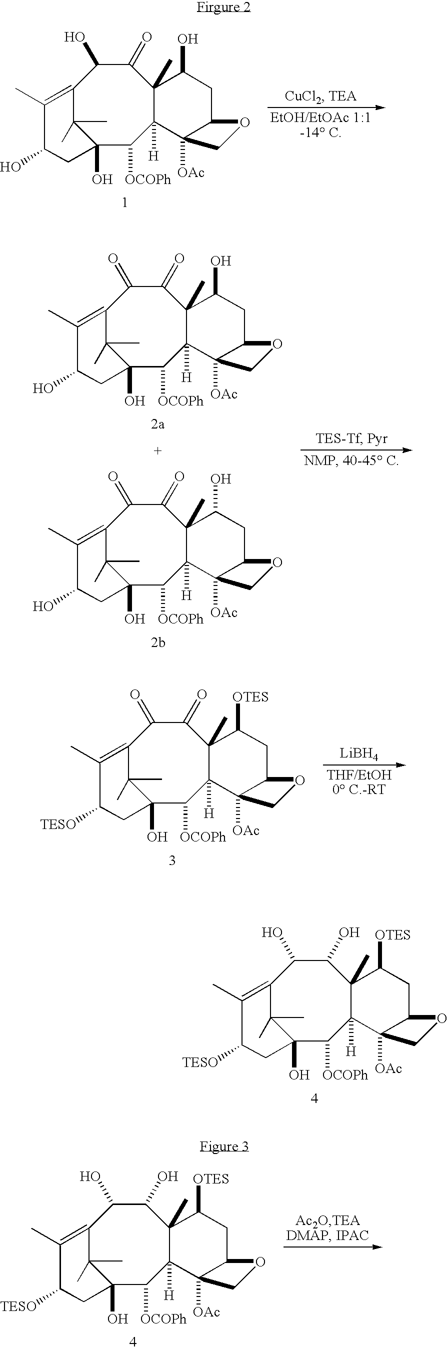

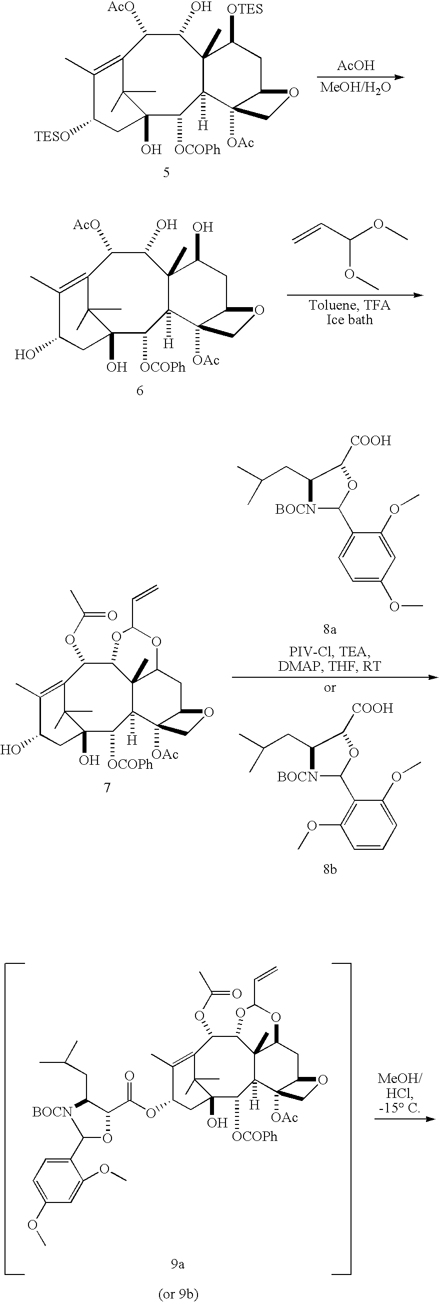

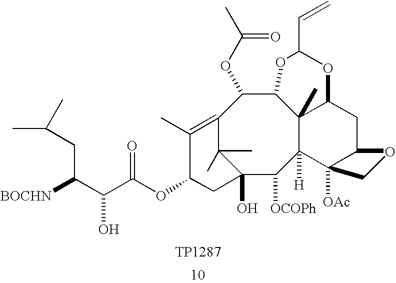

In one aspect as shown in FIG. 2, the 9-keto alcohol 1 is selectively oxidized to form the 9,10-di-keto 2: The di-keto 2 may be obtained as a mixture of the di-keto, 2a and 2b. In one variation of the process, the two isomers, 2a and 2b, may be separated to afford 2a, or the mixture may be used as is in the subsequent step without separation. The mixture may be derivatized to form the corresponding protected alcohol, and a number of applicable alcohol protecting groups are disclosed, for example, in T. W. Greene and P. G. M. Wuts, Protective Groups in Organic Synthesis, 3rd Edition, John Wiley & Sons, New York (1999). In one particular method, the mixture is derivatized to form the corresponding protected silyl ether, such as the triethylsilyl ether, such as by treating the mixture with TES-OTf (trifluoromethanesulfonic acid triethylsilylester), pyridine and solvents like NMP to form the 7,13-di-silyl ether 3. If desired, before the silylation step, the undesired isomer 2b may be separated from the desired isomer 2a using various methods known in the art, including column chromatography and crystallization. Alternatively, where a mixture of the isomers 2a and 2b are used as the starting mixture, the epimeric isomers of the corresponding di-silyl ether 3 obtained may be separated using standard procedures known in the art. Because the isomer 2b forms the di-TES ether at a slower rate than the isomer 2a, the reaction condition may be adjusted accordingly to favor the formation of the isomer 2a.

The di-silyl ether 3 may be reduced to the corresponding 9,10-di-ol 4. Reduction may be performed using a hydride reducing agent, such as using NaBH₄ in an organic solvent. In one method, reduction of the di-ketone may be accomplished using LiBH₄ in a solvent or solvent mixture, such as THF/EtOH to form the di-ol 4. The reaction may be performed at room temperature, or below room temperature, or at about 20° C. to about −10° C., more preferably at about 0° C.

In FIG. 3, the di-ol 4 is converted to the corresponding 10-acylated alcohol 5 using an acylation agent such as acetic anhydride, TEA, DMAP and IPAC, to form the 10-acylated alcohol 5. Selective hydrolysis of the TES groups may be accomplished using, for example, AcOH in MeOH/H₂O, or using IPAc/MeOH, to afford the tetra-ol 6. Acetalization of the 7,9-di-ol of compound 6, preferably using acrolein diethyl acetal in an organic solvent, such as toluene, and TFA in an ice bath, provides the allylidene acetal 7 in good yields. Coupling of the allylidene acetal 7 with the acid 8a affords the coupled product 9a. Deprotection affords compound 10 (TPI287) in good yields.

In another aspect, coupling reaction of the allylidene acetal 7 with the acid 8 affords the coupled product 9, which is not isolated, and the N,O-acetal is hydrolyzed in situ, as provided herein affords the product, compound 10 (TPI287) in good yields. The hydrolysis may be performed using an acid in an alcohol at low temperatures, such as hydrochloric acid in methanol at about −25° C. to 25° C., preferably about −15° C. This general procedure may be employed using either of the starting isomer 8a or 8b, that forms the corresponding isomer 9a or 9b, respectively.

As provided in FIG. 3, when the acid 8b is employed in the coupling reaction with compound 7, the resulting product 9b is formed as the coupled product.

The N,O-acetal 8b may be prepared according to the procedure illustrated in FIG. 4 to provide the desired product in good yield. Similarly, the N,O-acetal isomer 8a may be prepared according to the procedure illustrated in FIG. 4 to provide the product in good yield. A method which can be adapted for preparing the intermediates in FIG. 4 is disclosed in the Journal of Organic Chemistry, 2001, 66, 3330-3337, the reference of which is incorporated herein in its entirety.

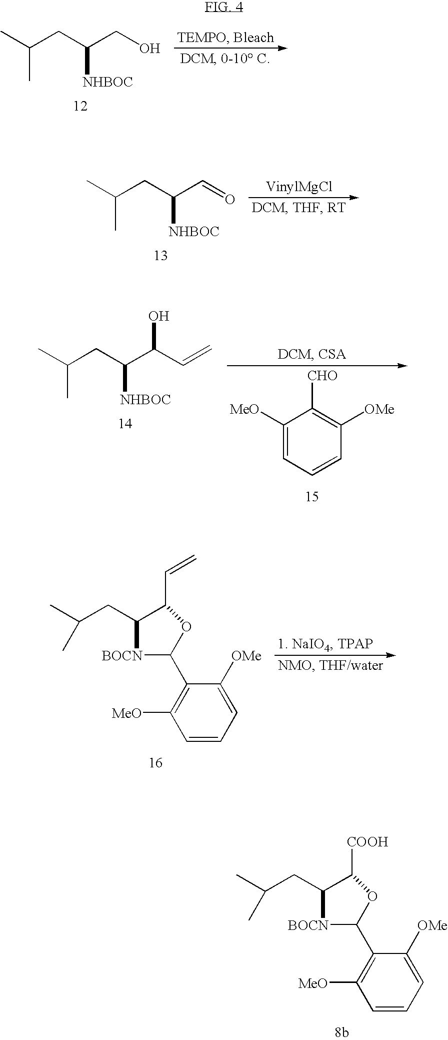

Depending on the desired purity of the intermediate(s) and the processing parameters that are used in the process, the intermediates described herein may be isolated and/or purified in one or more processing step before submitting to the subsequent reaction step or steps. In particular aspects of the process, depending on the desired purity, the reagents employed and the reaction conditions, the subsequent reaction step or steps of a reaction product (or intermediate) is subjected to one or more subsequent reaction without isolation and/or purification until the final product compound 10 (TPI287) is obtained. When desired, purification of the intermediates and/or product may be performed using various methods known in the art, including column chromatography, crystallization, distillation and the like, or the combination of the methods.

Equivalent protecting groups that may be used in the above cited procedures are known to one skilled in the art of organic synthesis. Such protecting groups, and the use of such groups in synthesis, may be found in various texts, including T. W. Greene and P. G. M. Wuts, Protective Groups in Organic Synthesis, 3rd Edition, John Wiley & Sons, New York (1999).

Standard procedures and chemical transformation and related methods are well known to one skilled in the art, and such methods and procedures have been described, for example, in standard references such as Fiesers' Reagents for Organic Synthesis, John Wiley and Sons, New York, N.Y., 2002; Organic Reactions, vols. 1-66, John Wiley and Sons, New York, N.Y., 2005; March J.: Advanced Organic Chemistry, 4th ed., John Wiley and Sons, New York, N.Y.; and R. C. Larock: Comprehensive Organic Transformations, Wiley-VCH Publishers, New York, 1999. All texts and references cited herein are incorporated by reference in their entirety.

### Example 1

### Oxidation of Vinylic Compound

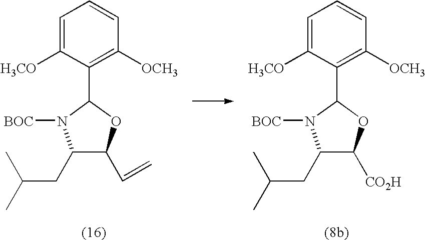

### Oxidation of Compound (16) to Compound of Formula (8b):

In a round bottom flask, NMO (10.49 g, 75.2 mmol) was stirred with ACN (200 mL) to obtain a solution. With stirring, to the solution was added 10% aqueous NaIO₄ (165 mL, 76.4 mmol), additional ACN (50 mL) and deionized water (50 mL). TPAP (504 mg, 1.4 mmol) was added after which a solution 16 (15.0 g, 38.3 mmol, 0.5 g/mL ACN) was added over the course of approximately 1 minute under ambient conditions. After ˜50 minutes additional ACN (50 mL), NMO (10.0 g, 71.7 mmol) and 10% aqueous NaIO₄ (82 mL, 38.0 mmol) were added to the reaction mixture to drive to completion. After reaction was completed, to the stirring reaction mixture was added. IPAc (300 mL) and water (200 mL). The mixture was vacuum filtered to remove precipitated reagents, and then it was partitioned. The aqueous phase was twice back extracted, once with IPAc and then with 2:1 n-heptane/IPAc. After each extraction the organic phases were combined.

After ensuring that the organic phase was slightly acidic, it was washed with 15% aqueous Na₂S₂O₃, followed by water and finally brine. The isolated organic phase was concentrated by rotary evaporation at 45° C. to give 9.82 g of crude 8b. The crude oil was purified by column chromatography to give 5.0 gm of 8b.

### Example 2

### Pivaloyl Mixed Anhydride

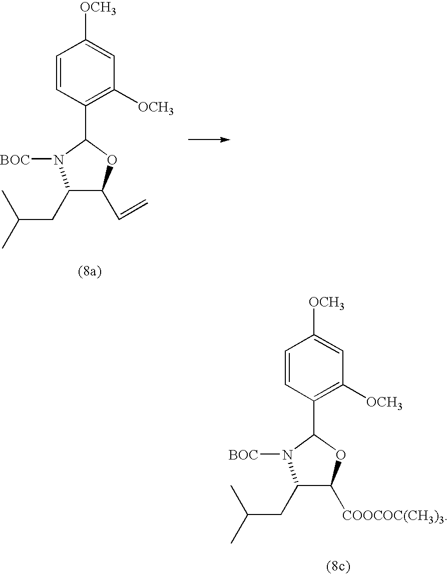

The sodium salt of the acid of formula (8a) may be obtained by the method of Bombardelli of al, WO 01/02407 or by neutralisation of the compound of Example 1.

A solution containing 55.00 g (127.5 mmol) of side chain, said sodium salt, in dichloromethane (550 mL) was washed with cold (0-5° C.) 2N aqueous HCl solution (2×460 mL). The organic phase was washed with 12.5 wt % sodium chloride solution (2×460 mL), dried over anhydrous sodium sulfate, filtered and concentrated in vacuo to constant weight to afford 50.35 g (96.5%) free acid, compound (8a).

To a 0-5° C. solution of 5.51 g (13.5 mmol) free acid compound (8a) in anhydrous THF (50 mL) under an inert atmosphere of nitrogen was added 1.78 mL (16.2 mmol) 99% 4-methylmorpholine and 1.99 mL (16.2 mmol) 99% trimethylacetyl chloride. The progress of the reaction was monitored by HPLC (a reaction aliquot was quenched into MeOH). After one hour, 0.20 mL (1.8 mmol, 0.2 eq) 99% 4-methylmorpholine and 0.22 mL (1.8 mmol, 0.2 eq) 99% trimethylacetyl chloride were added. After an additional 30 minutes at 0-5° C., the conversion, to the mixed anhydride side chain, compound (8c) was complete.

### Example 3

### Pivaloyl Mixed Anhydride and Use

The mixed anhydride of formula (8c) is prepared and employed in situ as follows

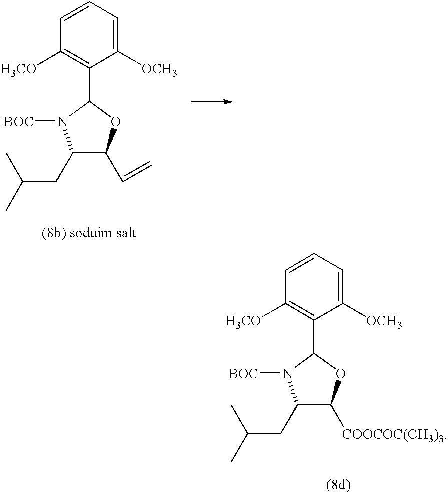

A 10 mL round bottom flask with two necks was heated to eliminate water, then allowed to cool under N₂ atmosphere. To the flask was added compound 7 (125 mg, 0.2 mmol) (as shown in FIG. 1), THF (1.25 mL), 4-methylmorpholine (40 μL, 0.36 mmol), DMAP (10.9 mg, 0.009 mmol), compound of formula (iii) (110 mg, 0.254 mmol) and finally trimethylacetyl chloride (40 μL, 0.319 mmol). The reaction mixture was stirred at 40° C. under N₂. After about 2 hours, additional 4-methylmorpholine (11 μL, 0.01 mmol), 8b sodium salt (41 mg, 0.1 mmol) and trimethylacetyl chloride (13 μL, 0.1 mmol) were added to assist formation of the anhydride intermediate which then coupled to 7. After about 2 additional hours, 4-methylmorpholine (11 μL, 0.010 mmol), trimethylacetyl chloride (13 μL, 0.104 mmol) and 8b sodium salt (42 mg, 0.1 mmol) were added. After 1.5 hours more, the reaction was placed into a freezer at −20° C. overnight. The following morning, stirring was resumed and the reaction was heated to 45° C. for 2 hours. Additional 4-methylmorpholine (22 μL, 0.02 mmol) and trimethylacetyl chloride (25 μL, 0.201 mmol) were added. An additional 2 hours of stirring resulted in the reaction reaching ˜90% completion.

To quench, the reaction mixture was removed from heat and allowed to cool to ambient. With stirring, MTBE (2 mL) was added followed by water (1 mL). The mixture was partitioned and the organic phase was washed with brine (40 μL). The organic phase was concentrated at 40° C. to obtain crude product as a pink foam.

The pink foam was dissolved into MTBE (500 μL) and added dropwise to stirring n-heptane (5 mL) at ˜−20° C. to give pink precipitate. The mixture was vacuum filtered and the solids were dried overnight in a vacuum oven at 40° C. to yield the desired coupled ester (82 mg), as indicated by LC/MS.

The coupled ester 9b was purified by flash chromatography on normal phase silica, eluting with an IPAc/n-heptane system of increasing polarity. Approximately 26 mg of the purified coupled ester was recovered as confirmed by LC/MS.

The coupled ester (15 mg, 0.001 mmol) was dissolved into THF (1 mL). A 250 μL aliquot of the solution was diluted 1:1 with THF. The solution was stirred on an ice bath at ˜0° C., after which HCl (0.5 N in MeOH, 25 μL) was added. The reaction was monitored by LC/MS, which indicated the formation of 10 (TPI 287). The depicted structure as shown is consistent by spectroscopic analysis using techniques like NMR and mass spectroscopy.

### Example 4

### Preparation of TPI 287

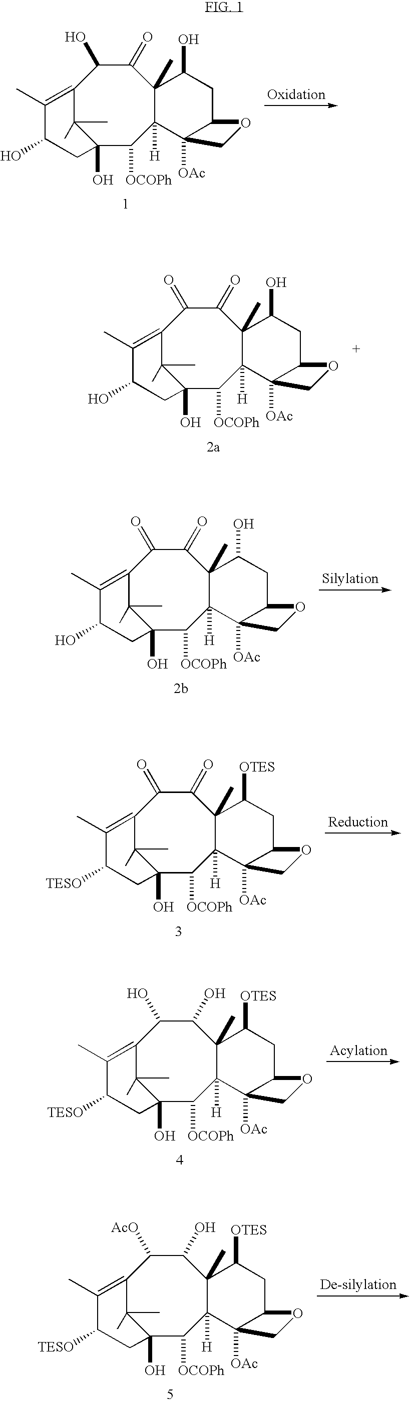

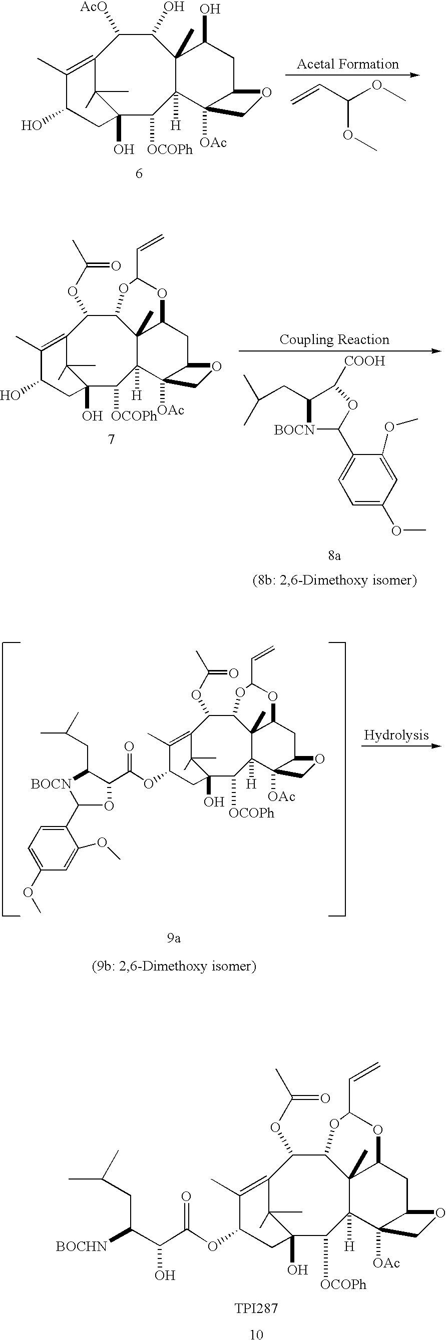

### I. Oxidation of 10 DAB III 1:

A 3 L round bottom flask (RBF), equipped with a magnetic stir bar was charged with 10 DAB III (25 g). EtOAc (700 mL) and EtOH (700 mL) were added, with stirring, until dissolution occurred. MgSO₄ was added and the mixture stirred for 2 hours. After sitting overnight, the mixture was filtered through celite (10 g) and the celite/MgSO₄ was washed with a solution of 1:1 EtOAc/EtOH (200 mL).

The combined filtrate was concentrated to a solid and placed in a vacuum oven at 45° C. for 24 hours to give dry 10 DAB III.

A 3 L round bottom flask (RBF), equipped with a magnetic stir bar and thermocouple and held under nitrogen, was charged with dried 10 DAB III (56.88 g, 104.85 mmol) followed by EtOH (710 mL, 12.5 mL/g) and EtOAc (570 mL, 10 mL/g). CuCl₂ was added to the suspension and the flask was placed in a NESLAB™ cooler. The reaction mixture was stirred and cooled to −13° C. A solution of TEA (51 mL, 366.97 mmol, 3.5 eq) in EtOAc (144 mL, 2.5 mL/g) was slowly charged to the flask while maintaining the temperature of the reaction at <−8° C. Stirring and cooling (≦−13° C.) continued until the reaction was complete as indicated by HPLC. The reaction was quenched with saturated ammonium chloride (156 mL) and EtOAc (600 mL) and cooling was discontinued.

Ammonium hydroxide (2M, 250 mL) was added and the reaction mixture was washed into a 4 L separatory funnel with water (250 mL) and EtOAc (300 mL). The mixture was further diluted with water (250 mL) and the layers were separated. The aqueous layer was back extracted with EtOAc (250 mL) and the organic layers were combined and washed with saturated ammonium chloride (3×250 mL). The organic layer was then washed with water (3×) and concentrated. Methanol (250 mL) was added, the mixture was stirred while heating to 50° C. for 1 hr. The mixture was then refrigerated for 2 hr, and filtered. The solids were collected and washed with methanol and dried in the vacuum oven (45° C.) to give 41.78 g of 2. Weight % yield=63.9%. As provided herein, the isolated product comprises approximately 10:1 to 20:1 of 2a to 2b.

### II. Tesylation of 2a to Form 3:

A 2 L round bottom flask equipped with a magnetic stir bar, a thermocouple and nitrogen bubbler was charged with 2a (45.0 g, 82.94 mmol) and IPAc (450 mL, 10 mL/g). The suspension was concentrated to solids on a rotavap at 45° C. To the solids were added 1-methyl-2-pyrrolidinone (270 mL, 6 mL/g). The mixture was placed in an ice/methanol bath and cooled to −11° C. Pyridine (40 mL, 6 eq,) was added to the reaction mixture and then cooled to −20° C. Triethylsilyl trifluoromethanesulfonate (TES-OTf) was added slowly to maintain the internal temperature at <−5° C. After the addition was complete, the flask was removed from the ice-bath. It was then placed in a warm-water bath and the reaction mixture was heated to 40° C. for ˜6 hours. The flask was transferred back into an ice-water bath, cooled to 5° C. and water (100 mL) was added dropwise to quench the reaction. The mixture was transferred to a 2 L separatory funnel and diluted with IPAc, heptane and water. The layers were separated and the aqueous layer was re-extracted with IPAc and heptane. The combined organic layers were washed with 10% CuSO₄, twice with water and then with brine. The mixture was filtered, concentrated and re-dissolved in n-heptane. This solution was concentrated to solids. n-Heptane was added to the solids and the suspension was concentrated on the roto-evaporator, the mixture cooled in the refrigerator, filtered and dried in a vacuum oven (45° C.) to give 48.27 g of 3 as a white solid. Weight % yield=79.5%.

### III. Reduction of 3 to Prepare 4:

A 1 L round bottom flask equipped with a magnetic stir bar and thermocouple and held under nitrogen was charged with THF (240 mL, 5 mL/g) and EtOH (240 mL, 5 mL/g) followed by 3 (48.0 g, 62.25 mmol). The reaction mixture was cooled in an ice/methanol bath to −13.9° C. A 2M solution of LiBH₄ in THF (62 mL, 2 eq) was slowly added to the flask while cooling and stirring to maintain a temperature ≦23° C. Cooling was stopped and the reaction continued to stir at room temperature until the reaction was complete as indicated by HPLC. The reaction flask was again placed in an ice/water bath and cooled to ˜2.5° C. and 10% NH₄OAc in EtOH (200 mL) was slowly added. Acetic acid (0.5 mL) was added and the solution was concentrated on a rotoevaporator. The concentrated solution was dissolved in methanol (200 mL) and added dropwise to stirring water. The resulting precipitate was filtered, washed with water and dried in a vacuum oven to give 46.34 g of 4 as a white solid. Weight % yield=72.4%.

### IV. Acetylation of 4 to Prepare 5:

A 1 L round bottom flask equipped with a magnetic stir bar and thermocouple was charged with IPAc (230 mL, 5 mL/g) followed by 4 (46.00 g, 59.34 mmol). The solution was concentrated to an oil on a rotoevaporator, to remove traces of water. The residual oil was re-dissolved in IPAc (200 mL) and DMAP (2.90 g, 0.4 eq), TEA (58 mL; 7 eq) and acetic anhydride (34 mL, 6 eq) were added. The reaction mixture was stirred under nitrogen at 36° C. until the reaction was complete as indicated by HPLC. Water (200 mL) was added to quench the reaction and the layers were separated. The aqueous layer was re-extracted with IPAc. The combined organic layers were concentrated to give 49.05 g of 5 as foam. Weight % yield=83.5%.

### V. Deprotection of 5 to Prepare 6:

A round bottom flask equipped with a magnetic stir bar and thermocouple was charged with methanol (360 mL, 10 ml/g) followed by 5 (36.5 g, 44.67 mmol). Acetic acid (75 mL, 2 mL/g) was added followed by the dropwise addition of water (70 mL, 2 mL/g). The reaction flask was placed in a warm water bath to dissolve the solids that formed when water was added and the reaction was stirred until complete as indicated by HPLC. Upon completion, the reaction was removed from the warm water bath and transferred to a 2 L RB recovery flask. A series of solvent exchanges with n-heptane (2×), 1:1 n-heptane/IPAc (3×) and heptane were performed. The final concentrated solution was applied to a silica pad and eluted under vacuum with 1:1 IPAc/heptane, IPAc and finally with EtOAc to give 28 g of 6 as foam, which was dissolved in toluene (1 L) at 50° C. The solution was concentrated to ˜150 mL and placed in a refrigerator overnight. The solids were filtered, washed with toluene (˜60 mL) and dried in the vacuum oven at 40° C. to give 21.3 g of 6 for a yield of 74.9%.

### VI. Acetal Formation: 6 to 7:

A round bottom flask equipped with a magnetic stir bar and thermocouple, was placed in a NESLAB™ Cooler at 0° C. and charged with 6 (700 mg, 1.885 mmol) and toluene (11 mL). TFA (274.6 μL, 3.0 eq) and acrolein diethyl acetal (365 μL, 2.0 eq) were added to the flask and the reaction was stirred until completion as indicated by HPLC. IPAc (11 mL, 15 mL/g) and 5% NaHCO₃ solution (6 mL) were added with stirring to quench the reaction. The reaction mixture was transferred to a separatory funnel and the layers were separated. The organic layer was passed over a normal phase silica pad (1.5 g, 2 g/g). The silica pad was eluted with IPAc. The combined filtrate was concentrated to dryness. The residual oil was re-dissolved in MTBE (3.5 mL). Heptane (˜3 mL) was added and the mixture was concentrated to give 640 mg of 7. Yield=78%.

VII. Preparation of Compound 10 (TPI287) from 7:

A round bottom flask equipped with a magnetic stir bar and thermocouple and held under nitrogen at room temperature was charged with 7 (3.042 g, 4.859 mmol), 8a (2.304 g, 1.1 eq), N-methylmorpholine (1.20 mL, 2.25 eq), DMAP (0.118 g, 0.2 eq) and anhydrous THF (50 mL). Pivaloyl chloride (1.2 mL, 2.0 eq) was added to the reaction mixture at room temperature and stirred until completion of the coupling reaction as indicated by HPLC. The solution was cooled in a NESLAB™ cooler (−18° C. to −20° C.) and 0.5 N HCl in methanol (20 mL) was added. The mixture was stirred at ˜−15° C. until the deprotection was complete as indicated by HPLC. Upon completion, the reaction mixture was quenched with 5% sodium bicarbonate solution (15 mL) and was concentrated to oil on a rotoevaporator. The residual oil was dissolved in methanol (4 mL) and added dropwise to water to precipitate 0.10 as solids. The solids were filtered, washed with water and dried in the vacuum oven overnight to give 4.0 g of 10. Yield=94%.

### Example 5

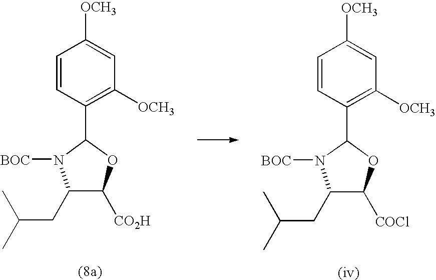

First, a solution was prepared containing 7.96 g (18.4 mmol, 3.0 eq) side chain, the sodium salt of acid (8a) and 2.25 g (18.4 mmol,) 99% 4-DMAP in anhydrous dichloromethane (80 mL). To this solution, 1.70 mL (19.1 mmol, 3.1 eq) 98% oxalyl chloride (neat) was added at ambient temperature under an inert atmosphere of nitrogen. The resulting mixture was stirred at ambient temperature for about 30 minutes, 98% oxalyl chloride (0.5 mL) was added and the mixture was stirred for an additional 30 minutes. HPLC analysis indicated conversion to acid chloride side chain, compound (iv) was complete (a reaction aliquot was quenched into methanol and analyzed as methyl ester). The mixture was filtered and the solids were washed with anhydrous dichloromethane (30 mL). The filtrate was concentrated under reduced pressure and the oil was further concentrated in vacuo under high vacuum for 25 minutes. The resulting oil was re-dissolved in anhydrous dichloromethane (30 mL) thereby producing a solution containing acid chloride side chain of compound (iv).

### Example 6

### Preparation of TPI 287

### Oxidation of 1:

A 4 L reaction flask, rinsed with dried EtOAc (300 mL) and held under N₂, was charged with dried EtOAc (1250 mL). Agitation was begun and dried 1 (100.04 g, 0.1837 mol) was added. The addition of USP EtOH (800 mL) followed and the reaction mixture was cooled to −1.3° C. (internal temperature). Anhydrous CuCl₂ (86.4 g, 3.5 eq) was added and solids from the sides of the flask were washed into the mixture with anhydrous EtOH (450 mL). The reaction mixture was cooled to −17.6° C. To maintain the internal temperature of the reaction at <−13° C., anhydrous TEA (90 mL, 3.5 eq) was added slowly. The reaction was monitored by HPLC/TLC. At 1 h the reaction was judged complete.

TFA (36 mL) was added to quench the reaction and stirring continued for 15 min. The reaction mixture was transferred into a 10 L rotovap flask. EtOAc (500 mL) and EtOH (300 mL) were added to the reaction flask, stirred for 2 min and the rinse added to the contents of the rotovap flask, which was evaporated on the rotovap at 40° C. until no further distillation occurred (80 min). Acidified ethanol (300 mL) was added to the residue and the resulting slurry was transferred to a 2 L rotovap flask. The first rotovap flask was rinsed into the second with acidified EtOH (400 mL). Again, the mixture was evaporated on the rotovap at 40° C. until no further distillation occurred (1 h). Acidified ethanol (305 mL) was added to the rotovap flask and the mixture was stirred on the rotovap at 40° C. for 10 min. The contents of the flask were then cooled to 5° C. and filtered. The rotovap flask was rinsed (2×) with cold (2° C.) acidified ethanol (300 mL) and the rinse was transferred completely to the filter to wash the solids. The solids were dried in the vacuum oven overnight at 45° C. to give 2. HPLC Area %=91.3%. Yield=96.72 g.

### TES Protection of 2:

To 2 (96.72 g, 0.1783 mmol) in a 10 L rotovap flask was added ethyl acetate (3000 mL, 30 mL/g). The solution was evaporated on the rotovap at 40° C. to approximately half the original volume (distilled volume=1680 mL). Toluene (1000 mL, 10 mL/g) was added to the remaining solution and it was evaporated on the rotovap at 40° C. until solids were obtained (45 min). The solids were suspended in toluene (1000 mL, 10 mL/g) and the suspension was evaporated on the rotovap at 40° C. (˜1 h) to dry solids. The solids were transferred to a 2 L flask equipped with a mechanical stirrer, thermocouple, addition funnel and N₂ stream (previously purged for 5 min). The solids in the rotovap flask were rinsed into the reaction flask with anhydrous pyridine (292 mL, 3 mL/g) and agitation was begun. Upon dissolution, agitation was continued and the contents of the flask were cooled to −20° C. Triethylsilyl trifluoromethanesulfonate (120.9 mL, 3.0 eq) was slowly added to the reaction mixture to maintain the internal temperature of the reaction at ≦−10° C. After the addition of TES-OTf was complete, the reaction mixture was allowed to warm to −5.8° C. and agitation continued. Thirty minutes after the addition of TES-OTf, sampling was begun and continued at thirty-minute intervals for HPLC/TLC. The reaction was judged complete at 2 h when HPLC/TLC indicated <2% of monoTES-2 remaining. The reaction mixture was cooled to −17.5° C. Methanol (19.3 mL, 0.2 mL/g) was added to quench the reaction and the reaction mixture was stirred for 5 min. While allowing the mixture to warm to ambient temperature, MTBE (500 mL) was slowly added with stirring and the mixture was transferred to a separatory funnel. Residues remaining in the reaction flask were washed into the separatory funnel with additional MTBE (200 mL, 2 mL/g), then water (250 mL, 2.5 mL/g) and saturated NH₄Cl solution (250 mL, 2.5 mL/g) were added. The mixture was agitated and the layers were separated. The organic layer was transferred to a clean container. MTBE (250 mL, 2 mL/g) was added to the aqueous layer. It was agitated and the layers were separated. The second organic layer was washed into the first organic layer with MTBE (100 mL) and water (200 mL, 2 mL/g) was added to the combined layers. This mixture was agitated and the layers were separated. The organic layer was transferred to a 2 L rotovap flask and evaporated to a residue at 40° C. n-Heptane (500 mL, 5 mL/g) was added to this residue and the solution was again evaporated to a residue at 40° C. n-Heptane (1000 mL, 10 mL/g) was added again and the solution was evaporated to ½ of its volume (distilled volume=375 mL). n-Heptane (300 mL, 2.5 mL/g) was added and the solution was stirred for 35 min on the rotovap at 40° C. The solution was then cooled to −15.7° C. while stirring was continued for ˜2.5 h. The solution was filtered. The solids remaining in the flask were rinsed into the filtration funnel with cold (<5° C.) n-heptane (100 mL) and all the solids were collected and dried overnight in the vacuum oven to give 111.22 g of 3. HPLC Area % purity=93.4%.

### Reduction of 3:

To THF (560 mL, 5 mL/g), stirring, and held under N₂ in a 4 L reaction flask, was added 3 (111.0 g, 0.144 mol) followed by anhydrous ethanol (560 mL, 5 mL/g). The mixture was stirred to dissolve the solids and then cooled to −12° C. 2 M LiBH₄ in THF (72 mL, 1.0 molecular, 4 chemical eq) was added slowly to control the reaction temperature (temp=−11.9 to −9.7° C.). The reaction mixture was stirred and sampled for HPLC/TLC at 30 min intervals. Additional 2 M LiBH₄ in THF was introduced slowly (72 mL, 1.0 eq) to the reaction flask (temp =−9.6° C. to −7.1° C.) and agitation continued for 30 min. A third addition of 2 M LiBH₄ in THF (36 mL, 0.5 eq) was made in the same manner as the previous additions (temp =−7.6° C. to −6.7° C.), but with the bath temperature adjusted to 15° C. following the addition of the LiBH₄ solution and to 12.5° C. ten minutes later. At 1 h following the final LiBH₄ addition, the reaction was judged complete. The reaction mixture was cooled to −10.8° C. and 10% ammonium acetate in EtOH (560 mL) was added slowly and cautiously to allow the foam to settle and to control the temperature of the solution ≦−3° C. The reaction mixture was transferred to a 2 L rotovap flask and any residues in the reaction flask were rinsed into the rotovap flask with EtOH (250 mL) and the contents of the rotovap flask were evaporated on the rotovap at 40° C. to an oil. Methanol (560 mL) was added to the residue. Water (1700 mL) was added to a 5 L flask equipped with an addition funnel and mechanical stirrer and was vigorously agitated. To precipitate the product, the methanol solution of the reaction mixture (748 mL) was slowly added to the flask containing water. The resulting mixture was filtered and the solids were washed with water (650 mL). A portion of the water was used to wash solids remaining in the precipitation flask into the filtration funnel. The solids were placed in the vacuum oven overnight at 45° C. to give 139.53 g of slightly wet product 4. HPLC area % purity=92.8%.

### Acetylation of 4:

To 4 (137.77 g, 0.178 mol) in a 2 L rotovap flask was added IPAc (1400 mL, 10 mL/g). The solution was evaporated on the rotovap at 40° C. to an oil (1.5 h). The procedure was repeated. Dried IPAc (550 mL) was then added to the residual oil and the contents of the rotovap flask were transferred to a 1 L reaction flask, equipped with a mechanical stirrer, addition funnel, thermocouple and a N₂ stream. The rotovap flask was washed into the reaction flask with IPAc (140 mL). DMAP (8.72 g, 0.4 eq), anhydrous TEA (170 mL, 7 eq) and acetic anhydride (100.6 mL, 6 eq) were added to the contents of the reaction flask and the mixture was stirred and heated to 35° C. While continuing agitation and heating to 35° C., the reaction was monitored by HPLC/TLC at 1-hour intervals.

Upon completion of the reaction, as indicated by the absence of 4 (3 h total reaction time), the reaction mixture was cooled to 19.7° C. and saturated ammonium chloride solution (552 mL) was added. After stirring for 15 min, the mixture was transferred to a separatory funnel, the layers were separated and the aqueous layer was removed. Water (280 mL) was added to the organic layer and the mixture was stirred for 4 min. The layers were again separated and the aqueous layer was removed. The organic layer was transferred to a 2 L rotovap flask and the remaining content of the separatory funnel was washed into the rotovap flask with IPAc (200 mL). The mixture was evaporated to dryness on the rotovap at 40° C. to give ˜124 g 5 as pale yellow oily foam.

### Deprotection of 5:

To the rotovap flask containing 5 (124.00 g) was added methanol (970 mL, 7 mL/g). Sampling for HPLC/TLC was begun and continued at 1-hour intervals. The 5/methanol solution was transferred to a 3 L reaction flask and agitation was begun. The remaining content of the rotovap flask was washed into the reaction flask with methanol (400 mL). Acetic acid (410 mL, 3 mL/g) and water (275 mL, 2 mL/g) were added and the reaction mixture was heated to 50° C. and stirred. With the temperature maintained between 50° C. and 55° C., the reaction was monitored by HPLC/TLC at 1-hour intervals for the disappearance of the starting material, formation and disappearance of the mono-TES intermediate and formation of the product 6. Upon completion (˜9 h), the reaction mixture was cooled to rt and transferred to a 10 L rotovap flask. Solvent exchanges to n-heptane (2×1370 mL, 1×1000 mL) and IPAc (2×1370 mL, 1×1500 mL) were performed. Then IPAc (280 mL, 2 mL/g) and silica (140 g, 1 g/g) were added to the rotovap flask and the contents were evaporated on the rotovap at 40° C. until no further distillation occurred and free flowing solids were obtained. The dry silica mixture was loaded onto a silica pad (7 cm column, 280 g silica), conditioned with 2:1 n-heptane/IPAc (500 mL, 2 mL/g silica) and washed (4×) with 2:1 n-heptane/IPAc (2 mL/g silica, 3400 mL total). Each wash (˜860 mL) was collected as a separate fraction and analyzed by TLC. The silica pad was again washed (4×) with 1:1 n-heptane/IPAc (3020 mL total, 2 mL/g silica) until all impurities were removed as indicated by TLC. Each wash (˜840 mL) was collected as a separate fraction and analyzed by TLC as before. The silica pad was then washed (5×) with waEtOAc (1% water, 1% AcOH in EtOAc) (3950 mL total, 2 mL/g silica) and with 1:1 MeOH/EtOAc and each wash (˜840 mL) was collected as a separate fraction. The product eluted with fractions 11-15. The fractions containing pure 6 as indicated by HPLC/TLC were combined, transferred to a rotovap flask and evaporated to dryness on the rotovap at 40° C. The residue in the flask was dissolved and evaporated to dryness: first with IPAc (1055 mL) and n-heptane (550 mL) and a second time with IPAc (830 mL) and n-heptane (410 mL). IPAc (500 mL) was then added to the residue, the solution Was transferred to a 2 L round bottom flask and n-heptane (140 mL) was added. The resulting solution was evaporated on the rotovap and dried in the vacuum oven at 40° C. to give 6 as foam. To dissolve the foam, IPAc (160 mL) was added to the flask followed by toluene (800 mL). The solution was evaporated on the rotovap under vacuum at 50° C. until half of the solvent was removed and solids were forming. The contents of the flask were stirred and cooled to 21° C. for 1.5 h. The solids were filtered in a 90 cm filtration funnel on #54 Whatman filter paper and were Washed with toluene (165 mL), transferred to the vacuum oven and dried at 40° C. to give 62.63 g of 6. HPLC area %=96.9%.

### Acetal Formation of 6:

Toluene (375 ml) was added to 6 (25 gm, 0.0424 mol) and cooled to ˜−15° C. TFA (9.8 ml, 3.0 eq) was slowly added when the slurry began to clear. The acrolein diethyl acetal (10.3 ml, 2.0 eq) was added and reaction was monitored every 30 min. Reaction was deemed complete when <3% 6 remained. 1 g/g hydrated silica (25% water) was added to quench the reaction at <˜−5° C.

Basic silica was added to the reaction mixture after 30-45 minutes while maintaining the reaction temperature <˜5° C. The pH of the reaction should be ˜5 by wetted pH paper. After stirring ˜15 min the silica was filtered off and washed with ˜20 ml/g toluene. The filtrates were combined and concentrated to ˜1 mL/g volume. The crystallization was held at room temperature for ˜4 hours and the solids were filtered and washed with minimal quantities of 80:20 Toluene:heptane to provide 17.6 gm of 7; HPLC area 98%.

### Coupling of 7 and Deprotection:

To THF (300 mL, 8 mL/g) stirring in a 1 L reaction flask (rinsed with THF (500 mL)) was added 7 (35.73 g, 0.0570 mol). Purified 8a (30.86 g, 1.25 eq) was added to the reaction mixture followed by the addition of NMM (11.5 mL, 1.8 eq), DMAP (2.77 g, 0.4 eq) and THF (75 mL, 2 mL/g). The mixture was stirred. Pivaloyl chloride (11.5 mL, 1.6 eq) was then added slowly to the reaction mixture. The reaction mixture was warmed and the temperature maintained at 38° C.±4° C. while stirring continued and N₂ continued to be bubbled from the bottom of the flask. The reaction mixture was analyzed by HPLC/TLC for consumption of starting material and formation of the coupled ester, 9a, at 30 min intervals beginning 30 min after the addition of the pivaloyl chloride.

After 1 h the reaction was judged complete and the reaction mixture was cooled to 2° C. 0.5 N HCl in MeOH (280 mL, 20 mL/mL of NMM used) was added to maintain the pH of the reaction mixture=1.5-1.9. The reaction mixture was stirred at 2° C.±2° C. and monitored by HPLC/TLC at 30 min intervals for consumption of 9a and formation of 10. Upon completion at 2 h the reaction was quenched with 5% aqueous sodium bicarbonate (300 mL) and IPAc (185 mL, 5 mL/g) was added. The reaction mixture was transferred to a 2 L rotovap flask and the reaction flask rinsed into the rotovap flask 2× with 60 mL IPAc. The mixture was evaporated under vacuum at 40° C. until a mixture of oil and water was obtained. IPAc (200 mL) was added to the oil and water mixture and the contents of the flask were transferred to a separatory funnel. The reaction flask was rinsed into the separatory funnel with IPAc (100 mL) and the contents of the separatory funnel were agitated and the layers were separated. The aqueous layer was removed. Water (70 mL) was added to the organic layer and, after agitation, the layers were separated and the aqueous layer was removed. The organic layer was transferred to a rotovap flask and evaporated under vacuum at 40° C. to a foam, which was dried in the vacuum oven to give 64.76 g crude 10. HPLC area % purity=45.5%.

The product was purified by silica gel chromatography with 65:35 n-heptane:wet acidified MTBE to give 41.74 g TPI 287. HPLC Area %=99.4%.

### Example 7

### Coupling Reaction

40 g of anhydrous sodium sulfate was added to a solution of C7, C10 di-Cbz 10-deacetylbaccatin III 5.00 g (6.15 mmol, 1.0 eq), in 150 mL dichloromethane. After three hours, the mixture was filtered and the filtrate was concentrated under reduced pressure. The C7, C10 di-Cbz 10-deacetylbaccatin III, was re-dissolved in anhydrous dichloromethane (50 mL) at ambient temperature, and subsequently, 2.25 g (18.4 mmol, 3.0 eq) 99% 4-DMAP was added and the solution was placed under an inert atmosphere of nitrogen. A solution of side chain, the acid chloride of Example 5, in dichloromethane, was added to the resulting solution at ambient temperature. The progress of the reaction was monitored by HPLC (a reaction aliquot was quenched into methanol). After stirring overnight, the solution was concentrated to dryness and the crude product was flash chromatographed over silica gel using 2/1 (v/v) EtOAc-heptane as the eluent. Appropriate fractions were pooled and concentrated in vacuo to constant weight to afford 7.31 g (98.7%) coupled product, as an off-white solid; 84.5 AP (230 nm).

### Example 8

### Coupling Reaction

A solution of the mixed anhydride of Example 2 (5.5 g, 13.47 mmol) in THF (30 mL) was cooled to 0° C. with an ice-water bath and 0.20 mL (1.8 mmol) 99% 4-methylmorpholine and 0.22 mL (1.8 mmol, 0.2 eq) 99% trimethylacetylchloride (pivaloyl chloride) were added. The reaction was stirred at ambient temperature for one hour. To this reaction mixture was then added a solution containing 1.76 g (14.4 mmol, 1.60 eq) 99% 4-DMAP and 7.30 g (8.98 mmol, 1.0 eq) of C7, C10 di-Cbz 10-deacetylbaccatin III, and the reaction was gently heated under reflux for about sixteen (16) hours under an inert atmosphere of nitrogen. After cooling to ambient temperature, the reaction was concentrated to dryness and reconstituted in EtOAc (60 mL). After stirring for about ten minutes, solids were removed by filtration. The filtrate was washed with saturated sodium bicarbonate solution (60 mL), water (60 mL) and brine (60 mL). The organic phase was concentrated to dryness to afford 14.52 g (>100%) crude coupled product. This crude material was dissolved into five volumes of MeOH and added dropwise (slowly) into water (10 volumes) with good stirring. The solids were filtered and dried to constant weight in vacuo at about 45° C. to yield 10.84 g (100%) coupled product, as a White solid; 74.2 AP (230 nm).

The reaction is repeated by replacing the mixed anhydride of Example 2 with that of Example 3.

### Example 9

### Coupling and Deprotection

The procedure of Example 6, Coupling and Deprotection, is repeated using compound 8b.

### Example 10

### Preparation of vinylic compound 16

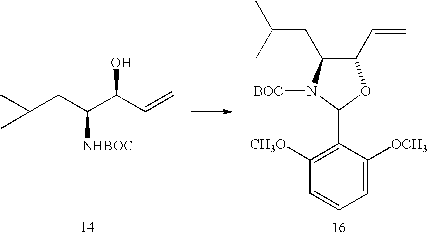

With magnetic stirring, 14 (90.0 g, 369.9 mmol) was dissolved in DCM (15 mL/g) and CSA (8.87 g, 36.99 mmol) was added. Lastly, 2,6-dimethoxybenzaldehyde (124.18 g, 747.1 mmol) was dissolved into the reaction mixture. The flask was purged with N₂ for several minutes and then the reaction mixture was brought to reflux for several hours until complete, as determined by TLC. The reaction mixture was removed from the heat and was quenched by the addition of 15% aqueous NaHCO₃ (300 mL). The mixture was partitioned and the organic layer was washed with water (100 mL). To the organic layer was added heptanes (1000 mL). Using a rotary evaporator, with mixing, the product solution was brought to 45° C. under slight vacuum to remove the DCM. As the DCM distilled, a precipitate began to form. The solution was placed into a −20° C. freezer overnight, continuing precipitation. The mixture was vacuum filtered to recover unreacted 2,6-dimethoxybenzaldehyde. The filter cake was rinsed with a minimum volume of heptanes. The filtrate was stripped at 45° C. to give yellow oil, crude 16 (118.77 g).

1. A compound of the formula (I): 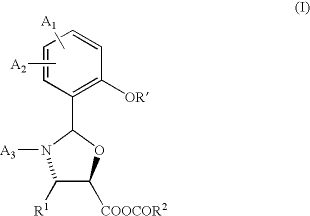
wherein
A1 is hydrogen, halogen, lower alkyl or lower alkoxy;
A₂ is hydrogen, halogen, lower alkyl or lower alkoxy;
A₃ is BOC or Cbz or PhCO;
R′ is methyl, ethyl or lower alkyl (C1 through C6);
R¹ is lower alkyl or phenyl group, and
R² is an alkyl or an aryl group such that the moiety OCOR² is readily displaced from the compound of formula (I) by an alcohol or alkoxide.

2. The compound of claim 1, wherein R′ is —OCH₃.

3. The compound of claim 1 wherein A₁ is a CH₃O group, aptly a 4- or 6-CH₃O group and preferably a 6-CH₃O group.

4. The compound of claim 1 or 2 wherein A₂ is hydrogen.

5. The compound of claim 1 of the formula (II) or (III) where in A₃ is BOC or Cbz:

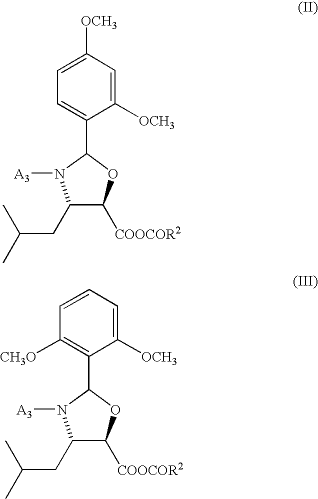

6. The compound of claim 1 of the formula (IV) or (V): where in A3 is BOC or Cbz:

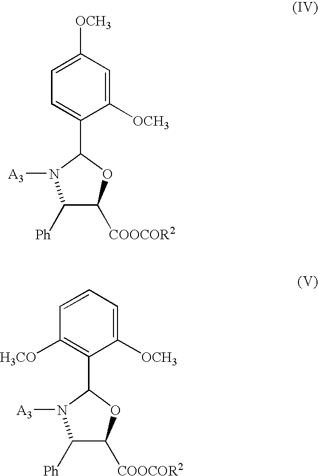

7. The compound according to claim 1 wherein R² is a C(CH₃)₃ group.

8. A compound of the formula (VI):

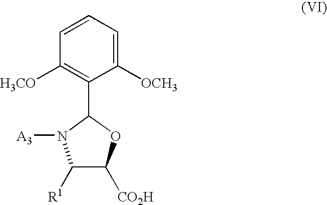

9. The compound of claim 7 wherein A₃ is selected from the group consisting of BOC, Cbz and PhCO and R¹ is phenyl.

10. An acid fluoride or acid chloride of a compound as claimed in claim 7.

11. A compound of the formula (I): 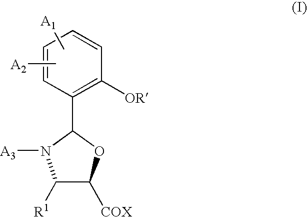
wherein
A₁ is hydrogen, halogen, lower alkyl or lower alkoxy;
A₂ is hydrogen, halogen, lower alkyl or lower alkoxy;
A₃ is BOC or Cbz or PhCO;
R¹ is methyl, ethyl or lower alkyl (C₁ through C₆);
R¹ Is lower alkyl or phenyl group, and
X is a halide selected from the group consisting of F, Cl, Br and I.

12. The compound of claim 11, wherein A₂ is 4-alkoxy and A₃ is 6-alkoxy.

13. The compound of claim 12, wherein A₂ is 4-methoxy and A₃ is 6-methoxy.

14. The use of a compound as claimed in claim 1 in the acylation of a 13-hydroxy group on a taxane derivatives.

15. A compound of the formula (IA): 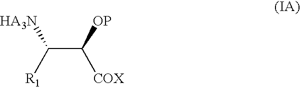
wherein
A₃ is BOC or Cbz or PhCO;
P is a hydroxyl protecting group;
R¹ is lower alkyl or phenyl group, and
X is a halide selected from the group consisting of F, Cl, Br and I.

16. A compound of the formula (VII): 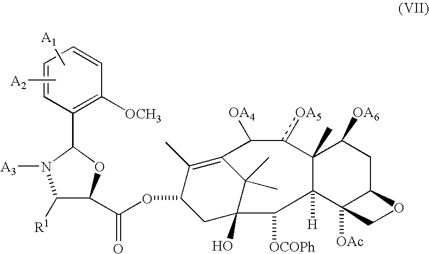
wherein
A¹, A², A³ and R¹ are as defined in claim 1,
A₄ is a hydrogen atom or is a hydroxyl protecting group selected from the groups consisting of benzyl, Cbz and acetyl group;
A₅ is
(i) absent when the dotted line represents a second bond to the oxygen atom so that [symbol] OA₅ is an oxo group or
(ii) a hydrogen atom or
(iii) a Cbz group or
(iv) joined to A₆ to form OCH(CH═CH₂)O group.

17. A compound of claim 16 of the formula (VII) wherein:
A₄ is acetyl and OA₄ is in the α configuration, and
A₅ is joined to A₆ and OA₅ is in the α configuration.

18. A compound of claim 16 wherein OA₄ has a β configuration and A₄ is hydrogen, Cbz or acetyl, [symbol] OA₅ is an oxo group; and A₆ is hydrogen or Cbz.

19. A process for the preparation of paclitaxel, docetaxel or TPI 287 which comprises the hydrogenation of a corresponding compound of the formula VII as defined in claim 16.

20. The process for the preparation of a compound as claimed claim 1 wherein the corresponding carboxylic acid is reacted with a compound of the formula R²COCl or R²COF.

21. A process of claim 20 which employs (CH₃)₃CCOCl.

22. A process for the preparation of a compound of the formula VIII: 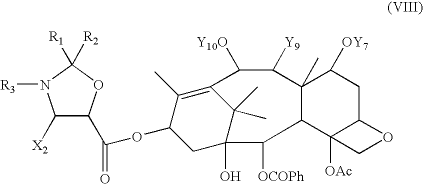
which compromises the reaction of a compound of the formula (IX): 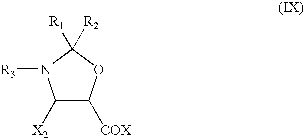
with a compound of the formula (X): 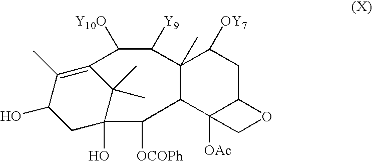
wherein:
R₁ and R₂ are independently H or substituted or unsubstituted alkyl, alkenyl, aryl, aralkyl or acyl;
R₃ is H or P₁ where P1 is an amino protection group;
X is halogen or OR⁴ where R⁴ is H, a substituted or unsubstituted alkyl, alkenyl, aryl, aralkyl, acyl, acyloxycarbonyl or aryloxycarbonyl;
X₂ is substituted or unsubstituted alkyl, alkenyl, aryl, aralkyl or acyl; Y₇ is R₇, P₃ or Z₇;
Y₉ is H, OH, a ketone, OR, P₄ or Z_{g};
Y₁₀ is R₁₀, P₅ or Z₁₀;
R₇ is H, substituted or unsubstituted alkyl, alkenyl, aryl, aralkyl or acyl;
Z₇ is P₃ and together with Y₉ forms a cyclic structure when Y₉ is P₄;
Z₉ is either R₉ and together with Y₇ forms a cyclic structure when Y₇ is P₃; or
Z₁₀ is P₅ and together with Y₉ form a cyclic structure when Y₉ is P₄;
P₅ and together with Y_{g} forms a cyclic structure when Y₁ is P₄;
R₉ is a substituted or unsubstituted alkyl, alkenyl, aryl, aralkyl or acyl;
R₁₀ is H, substituted or unsubstituted alkyl, alkenyl, aryl, aralkyl or acyl;
P₃ is a hydroxyl protecting group;
P₄ is a hydroxyl protecting group; and
P₅ is a hydroxyl protecting group.

23. The process of claim 22 wherein X is fluorine or chlorine, preferably fluorine.

24. The process of claim 22 wherein X is OCOR² where R² is as defined in claim 1.

25. The process of claim 22 wherein X is a OCOC(CH₃)₃ group.

26. The process according to claim 22 wherein the compound of formula (IX) is of the formula (XI): 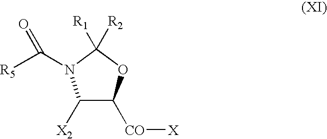
wherein
X₂ is phenyl or CH₂CH(CH₃)₂:
R₅ is (CH₃)₃CO or Ph or PhCH₂O
R₁ and R₂ are independently hydrogen, lower alkyl, lower alkyl substituted by lower alkoxy, phenyl or phenyl substituted by one, two or three groups selected from lower alkyl, lower alkoxy, fluorine or chlorine.

27. The process according to claim 26 wherein R₁ is hydrogen.

28. The process according to claim 26 wherein R₂ is an optionally substituted phenyl group.

29. The process according to claim 28 wherein R₂ is of the formula: 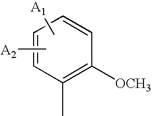
wherein A₁ and A₂ are as defined in claim 1.

30. The process according to claim 22 wherein the compound of the formula (X) is of the formula (XII):

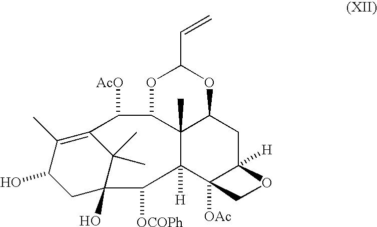

31. The process according to claim 22 wherein the compound of the formula (X) is of the formula (XIII): 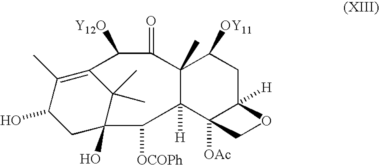
wherein Y₁₁ is hydrogen or a hydroxyl protecting group and Y₁₂ is hydrogen, acetyl or a hydroxyl protecting group.

32. The process of claim 31 where Y₁₁ is a protecting group removable by hydrogenation, preferably a Cbz group, and Y₁₂ is an acetyl group or a hydroxyl protecting group or a protecting group removable by hydrogenation, preferably a Cbz group; or wherein Y₁₁ and Y₁₂ are independently protecting group that may be cleaved by non-hydrogenolysis methods.

33. The compound of the formula VIII as defined in claim 22.

34. The compound of claim 33 of the formula (XIV): 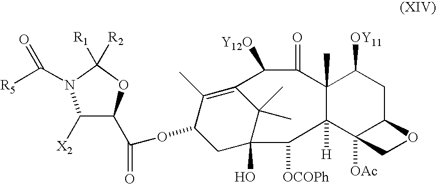
wherein R₁, R₂, R₅ and X₂ are as defined in claim 26 and wherein
(a) Y₁₁ is hydrogen or a hydroxyl protecting group and Y₁₂ is hydrogen, acetyl or a hydroxyl protecting group; or
Y₁₁ is a protecting group removable by hydrogenation, preferably a Cbz group, and Y₁₂ is an acetyl group or a hydroxyl or a protecting group removable by hydrogenation, preferably a Cbz group; or
(c) Y₁₁ and Y₁₂ are independently protecting group that may be cleaved by non-hydrogenolysis methods.

35. A compound of the formula (XV): 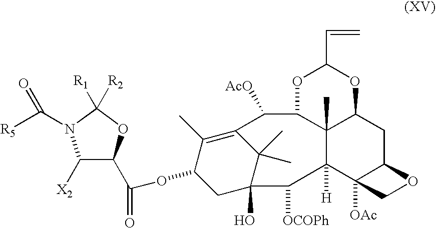
wherein R₁, R₂, R₅ and X₂ are as defined in claim 26.

36. The use of a compound of the formula VIII as defined in claim 22 as an intermediate in the manufacture of a taxane derivative.

37. A process for the preparation of a compound of the formula (XVI) 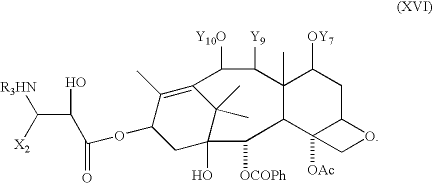
wherein X₂, R₃, Y₁₀, Y₉ and Y₇ are as defined in claim 22 which comprises deprotecting the protecting groups in a compound of formula (VIII).

38. A process according to claim 37 wherein the compound of the formula (VII) is a compound of the formula (XV) as defined in claim 35.

39. A process for the preparation of a compound of TPI 287 comprising:
a) selective oxidation of keto-alcohol 1 to afford compound 2a;
b) protection of the 7,13-di-hydroxy compound 2a to afford compound 3;
c) selective reduction of compound 3 to provide di-ol 4;
d) derivatizing di-ol 4 to form ester 5;
e) deprotection of the protected ethers to form tetra-ol 6;
f) acetalization of tetra-ol 6 to form acetal compound 7;
g) coupling of compound 7 with compound 8a to afford compound 9a; and
h) deprotection of compound 9a to form compound 10 (TPI1287) as shown in the FIG. 1, below:

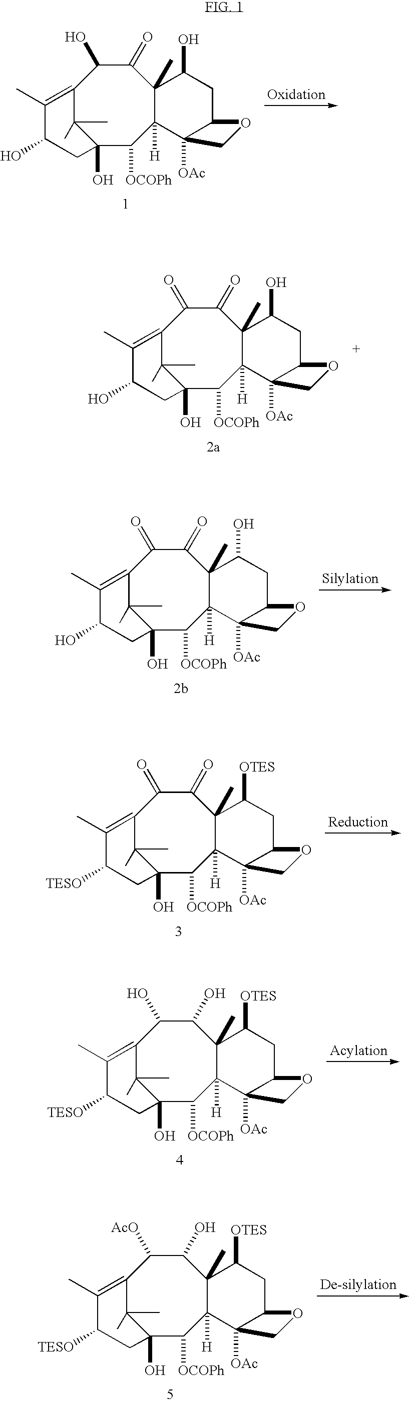

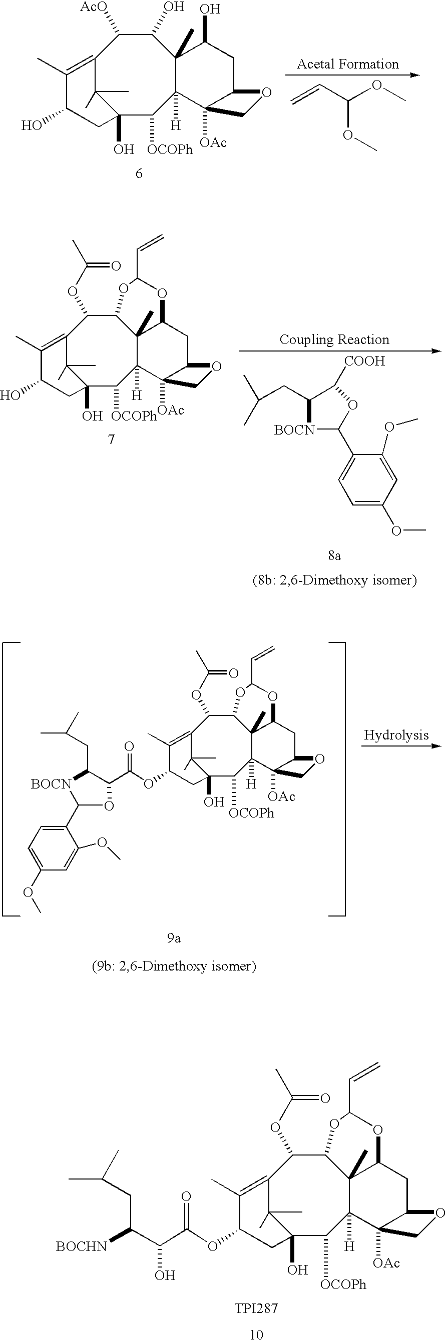

40. A process for the preparation of TPI287 comprising:
a) selective oxidation of keto-alcohol 1 to afford compound 2;
b) protection of the 7,13-di-hydroxy compound 2 to afford compound 3;
c) selective reduction to provide di-ol 4;
d) derivatizing di-ol 4 to form ester 5;
e) deprotection of the silyl ethers to form tetra-ol 6;
f) acetalization of tetra-ol 6 to form compound 7;
g) coupling of compound 7 with compound 8a to afford compound 9a; and
h) deprotection of compound 9a to form TPI₂₈₇, compound 10, as shown in FIGS. 2 and 3;

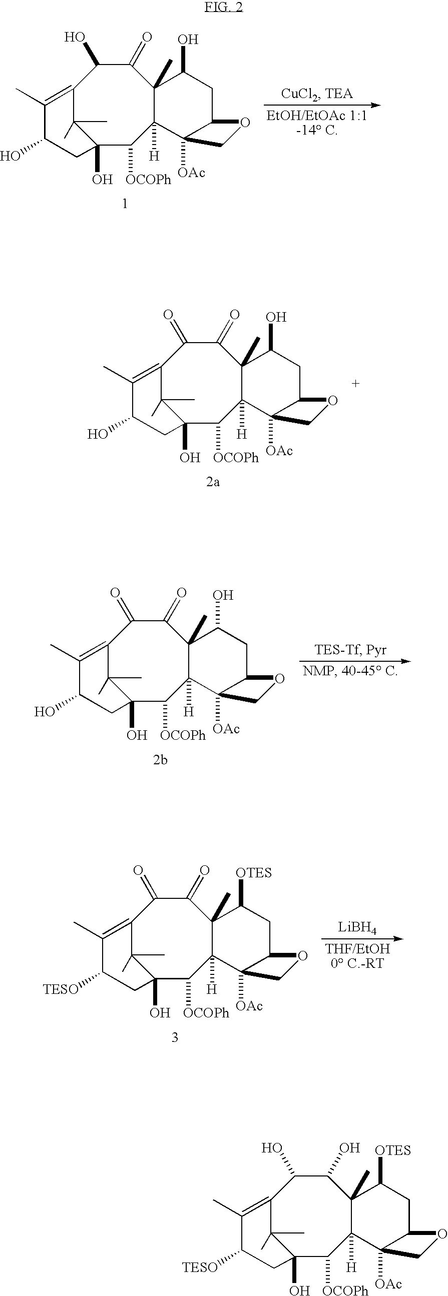

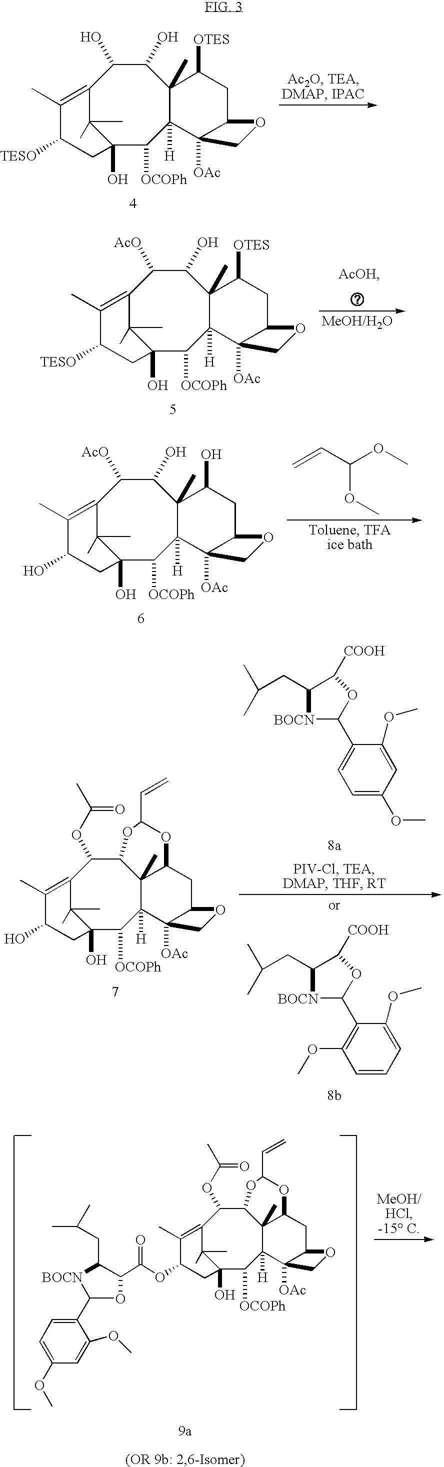

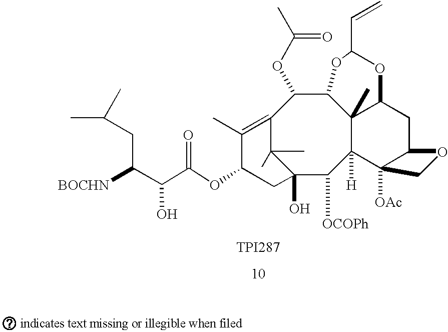

41. A process for the preparation of a compound of the formula (XVI): 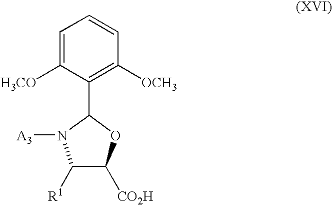
wherein R1 is lower alkyl or phenyl and A3 is BOC or Cbz which comprises the oxidation of a compound of the formula (XVII):

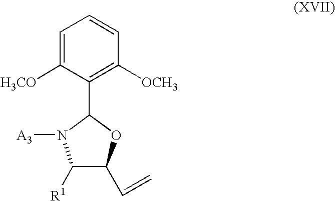

42. A compound of the formula (XVII) as defined in claim 41.

43. A process for the preparation of compound 8b, as described in FIG. 4.

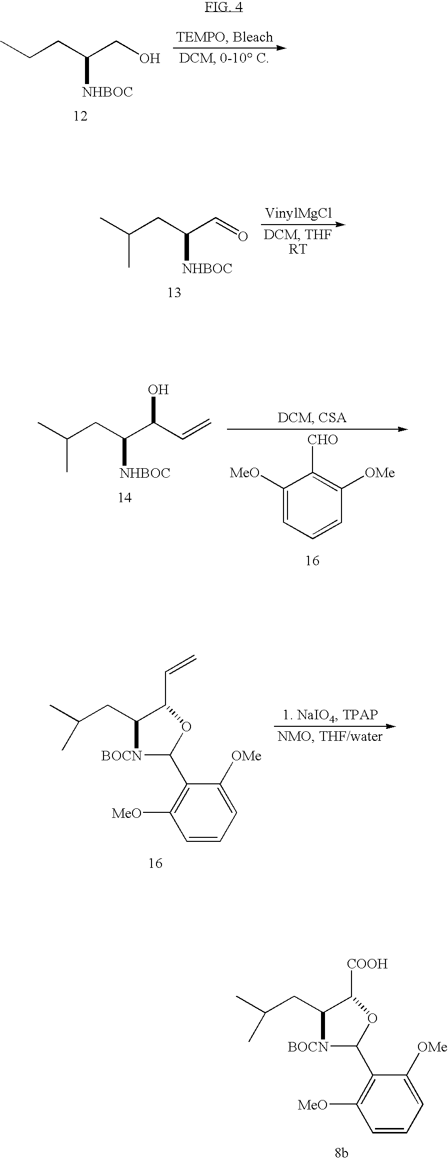

44. A process analogous to that of claim 43 wherein the 2,4-dimethoxy compound is used in place of the 2,6-dimethoxy compound 15.

45. A process for the preparation of compound 9b as shown in FIG. 5: [chemical structure]
